(19) United States
(12) Patent Application Publication
(10) Pub. No.: US 2003/0092957 A1
(43) Pub. Date: May 15, 2003

(54) Brachytherapy device and method of use

(76) Inventors: Neal A. Scott, Stone Mountain, GA (US); Janet M. Hampikian, Decatur, GA (US)

Correspondence Address: Finnegan, Henderson, Farabow,, Garrett & Dunner LLP, 1300 I Street, N.W., Washington, DC, 20005-3315, US

(21) Appl. No.: 10/112,764

(22) Filed: Apr. 2, 2002

Related U.S. Application Data
(63) Continuation-in-part of application No. 09/386,779, filed on Aug. 31, 1999, now Pat. No. 6,475,644.
(60) Provisional application No. 60/280,136, filed on Apr. 2, 2001.
(60) Provisional application No. 60/108,963, filed on Nov. 18, 1998.
(60) Provisional application No. 60/141,766, filed on Jun. 30, 1999.

(51) Int. Cl. C23C 18/00; C23C 20/00; A61N 5/00

(52) U.S. Cl. 600/3; 428/457; 428/680; 428/686; 428/926; 428/655; 106/1.26; 427/405

(57) ABSTRACT

The present invention is directed to a brachytherapy device comprising a substrate comprising at least one radioactive coating layer formed thereon. The radioactive coating layer has a total radioactivity that varies in at least one dimension of the device. Methods of making radioactive coatings, such as electrochemical deposition, electroless deposition and sol-gel are also disclosed. Suitable substrates include medical devices, such as catheters, stents, brachytherapy devices and guidewires, or components thereof. The disclosed methods produce medical devices capable of generating asymmetric, or targeted, radiation fields that correspond to the morphology of a tumor. Methods of using these devices to treat cancer of the breast, brain, prostate, uterine, head and neck are also disclosed.

This application claims the benefit of priority under 35 U.S.C. §119(e) of Provisional Application No. 60/280,136, filed Apr. 2, 2001, and is a continuation-in-part of application Ser. No. 09/386,779, filed Aug. 31, 1999, which claims the right of priority under 35 U.S.C. §119(e) of Provisional Applications No. 60/108,963, filed Nov. 18, 1998, and No. 60/141,766, filed Jun. 30, 1999, the entire contents of each of which are hereby incorporated by reference.

### BACKGROUND OF THE INVENTION

1. Field of the Invention

The present invention relates to radioactive coating solutions, radioactive sols and sol-gels, methods used to form radioactive coatings on a variety of substrates, and to radioactive coated substrates. In particular, the present invention relates to a medical device, or a component thereof, having at least one radioactive coating layer thereon. The medical device is preferably a device for treating tumors. More preferably, the invention is directed to a device for brachytherapy for solid tumors and the cavities from which tumors have been removed.

2. Description of Related Art

Brachytherapy is the treatment of cancer by radioactive elements placed at a short distance from a tumor. This technique was born shortly after Henri Becquerel's discovery of radioactivity in Paris in 1860. After Marie Curie extracted a tiny amount of radium from tons of pitchblend ore, she and her husband Pierre Curie loaned a small radium tube to Danlos at St. Louis Hospital in Paris, who treated a patient with lupus. In 1903, Goldberg and London successfully treated two patients in St. Petersburg suffering from basal cell carcinoma of the face. In 1905, Robert Abbe, chief surgeon at St. Luke's Hospital, New York, obtained two tubes containing radium from the Curies in Paris and used it as an adjuvant to surgery. After resection of a tumor, he positioned tubes into the tumor bed and later inserted the radium sources, thus pioneering the afterloading technique of radium therapy in the United States.

By 1915, Janeway was using interstitial radium needles for the treatment of primary breast cancer. (Hilaris, B. S., Mastoras, D. A., Shih, L. L., Bodner, W. R., History of Brachytherapy: The Years After the Discovery of Radium and Radioactivity, Principles and Practice of Brachytherapy, p. 13 (S. Nag ed., Futura Publ., (1997)). In the 1920s, Keynes began using radium needles in one of the first published series of breast conservation therapy (Keynes. G., The Treatment of Primary Carcinoma of the Breast with Radium., Acta Radiol. 10, p.393-402 (1929)). Keynes placed radium needles within the entire breast, and within the internal mammary, supraclavicular, infraclavicular, and axillary lymph nodes. He reported high local and regional control rates, acceptable cosmetic outcome and survival rates which were stated to be as favorable as the Halstead radical mastectomy.

Despite these positive results, for the next 30 years breast cancer was treated predominantly by mastectomy. During the 1970s, a new approach to breast conservation therapy was inspired by Vera Peters and others. The new concept was a surgical removal of the tumor within the breast, and of the axillary lymph nodes, followed by modest doses of radiation therapy to the entire breast to sterilize subclinical disease. During this time, cobalt-60 teletherapy and low energy photon beam therapy were available, but not electron beam therapy. Therefore, the method most often used to deliver supplemental dose to the vicinity of the tumor excision site was brachytherapy. Typically, 50 Gy in 25 fractions was delivered to the entire breast and supraclavicular nodes, followed by an interstitial brachytherapy boost of 15 to 20 Gy with low dose rate iridium-192.

In the 1980s, electron beam radiation therapy became widely available and quickly supplanted brachytherapy as the preferred boost method. The necessity of routine boosting after whole breast radiotherapy became controversial when the NSABP trial B-06 was published. In this study, no boost at all was given in the radiation therapy arm when negative surgical margins were required. This change in practice pattern resulted from several factors. Electron beam ports are easy to set up, deliver and are non-invasive. The radiation oncologist could select from an array of energies, choosing a beam that did not penetrate deeply into the lung or heart.

Currently, most radiation oncology centers reserve interstitial brachytherapy boost for patients with large breasts and deeply seated tumors, for whom the integral dose with the external beam radiation therapy would be significantly greater than that with brachytherapy. Patients with microscopically positive margins or with residual gross tumor are also excellent candidates for brachytherapy boost. Thus, brachytherapy has become the primary method of breast irradiation in only select breast cancers.

Studies have shown that a vast majority of local breast cancer recurrences occur in the vicinity of the surgical excision site, both with whole breast irradiation and without. In either case, no more than 3.5% of patients experienced recurrence in remote locations of the breast. Therefore, one could hypothesize that the primary benefit of breast irradiation comes from radiation directed at the 2 cm of tissue surrounding the excision cavity, and that the entire breast does not need to be treated in every case. Furthermore, certain features of breast cancer have been shown to be predictors for a higher risk of remote relapse. Appropriate selection criteria may allow breast brachytherapy to be the sole method of breast irradiation, without a significant risk of remote relapse. The following criteria may be used to select patients for breast brachytherapy:

(1) T1 and T2 tumors less than 3 cm in greatest gross and microscopic dimension;

(2) Negative inked microscopic surgical margins;

(3) No invasive or in-situ lobular carcinoma;

(4) Three or fewer metastatic axillary lymph nodes, without extracapsular extension;

(5) No collagen vascular disease;

(6) No treatment with neoadjuvant chemotherapy;

(7) No longer than 6 weeks time interval since definitive breast surgery;

(8) Unifocal breast cancer, without diffuse suspicious microcalcifications, with no known unresected residual carcinoma, and with a negative post-tylectomy mammogram; and

(9) No extensive intraductal carcinoma.

With some of the exceptions discussed above, for the past 100 years breast cancer therapy has primarily been based on the premise that treatment of the entire breast is necessary for local control and disease-free survival. With mastectomy, the whole breast is treated by removing it. With breast conservation therapy, the whole breast is treated with external beam radiation therapy following limited excision of the tumor.

Breast conserving therapy has become an accepted option in the treatment of most patients with Stage I and Stage II breast cancer. Multiple retrospective studies and seven prospective randomized trials have established the equivalence of this treatment approach compared to mastectomy in terms of disease free and overall survival. The major advantage of breast conserving therapy relates to the superior cosmetic result and reduced psychological and emotional trauma resulting from this procedure, compared to mastectomy.

Breast conserving therapy, however, also has relative disadvantages. The technique is a more complex and prolonged treatment regimen that requires approximately 5-7 weeks to complete. As a result, for patients who are elderly or live a significant distance from treatment centers, transportation problems can prove prohibitive. In addition, with the more frequent use of adjuvant systemic chemotherapy in both lymph node-negative and -positive patients, substantial delays can be incurred prior to the initiation of either local breast irradiation or systemic chemotherapy.

Despite the obvious cosmetic and emotional advantages of breast conserving therapy, only 10-40% of patients who are candidates for breast conservation actually receive it. (Farrow D. C., Geographic Variation in the Treatment of Localized Breast Cancer, New England Journal of Medicine, 326:1097-1101,1992). The reasons for the underuse of this treatment approach are multifactorial, related in part to physician bias, and/or to the logistics and time involved with the delivery of radiation therapy. (Fisher, B., On the Underutilization of Breast Conserving Surgery for the Treatment of Breast Cancer, Ann. Oncol., 4:96,1993).

Most of the logistical problems with breast conserving therapy relate to the protracted course of external beam radiation therapy to the whole breast. Standard therapy after tumor excision generally includes 5 weeks of external beam radiation therapy to the whole breast followed by a boost to the tumor bed with either an additional 8-10 fractions (days) of external beam radiation therapy or a 2-3 day interstitial implant. (Vincini F, et al. The Role of Total Dose in Conservative Surgery and Radiation Therapy for Early Stage Breast Cancer: Is There a Critical Level? IJROBP32:257, 1995). The rationale for this approach is based on two principles.

First, higher doses of XRT are given to the “tumor bed” in an attempt to control residual small foci of cancer that may be left behind after the excision. Second, whole breast external beam radiation therapy is used to eliminate possible areas of occult multicentric, in situ or infiltrating cancer in remote areas of the breast. That such remote, multicentric areas of cancer exist has long been established. However, the biological significance of these areas of occult cancer is unknown and the necessity to treat the entire breast prophylactically has recently been questioned.

In addition to breast cancer, other types of malignant tumors make cancer the second leading cause of death in the U.S. In those patients who are diagnosed with cancer, common treatments include surgical removal of the tumor, chemotherapy and radiation therapy. As illustrated for breast cancer, patients are frequently treated with a combination of these methods, which include surgery to remove the tumor. In some circumstances, however, removal of the tumor is not an option. Brachytherapy is used in such circumstances.

Conventional brachytherapy devices consist of needles through which radioactive sources are placed. The needles are used on a temporary basis and are removed after the radiation exposure. This technique can include both rigid or flexible tubes and other similar structures. Because of the size of these devices, the brachytherapy session is usually limited to several hours. Since the time of radiation is limited, the amount of radiation used must be relatively high. The high dose of radiation associated with this procedure increase the likelihood of radiation toxicity.

As a result, other devices have been developed for permanent implantation of tumors. The advantage of this method is that the doses of radiation can be much smaller since the exposure time is very long. The prototype device for this type of radiation delivery is the radioactive seed. These seeds are the size of a grain of rice and are composed of either ¹⁰³Pd or 125I covered by a layer of metal. The seeds are implanted into a tumor under an x-ray or ultrasound guidance and left permanently in place. This is the major type of radiation treatment for prostate cancer, the second most common cause of cancer in men.

A major disadvantage of implanting radioactive seeds is the relatively inaccuracy in the placement of the seeds. Even when seeds are placed appropriately placed, they can migrate. As a result, the dose given to the tumor is potentially sub-optimal and/or the dose delivered to the normal tissue, e.g., urethra, may be significantly higher than intended. In addition, seeds do not exhibit the ability to tailor a radiation field to a particular patient and/or tumor.

Other devices aimed at site-specific cancer treatment systems involve a balloon filled with radioactive liquid for treatment of brain or breast tumors. The device is a balloon catheter that is placed in the tumor excision site. The balloon is then filled with a radioactive liquid (such as Rhenium-188) and left in place for several days. A disadvantage of this type of treatment is the potential for a spill of radioactive liquid during instillation or withdrawal of the solution.

A comparison of the properties and uses between the present invention and other implantable brachytherapy devices for site-specific cancer treatment is given in Table 1.

TABLE 1
Comparative Devices for Site-Specific Cancer Treatment

| | Present | Comparative | Comparative |
|---|---|---|---|
| | Invention | System | System |
| Mechanism | Coated Wires | Balloon implanted | Implanted seeds |
| | | with radioactive | containing ¹⁰³Pd |
| | | liquid | |
| Sites | Brain, Breast, | Brain, Breast | Prostate, |
| | Prostate, Head | | Breast (?) |
| | and Neck, | | |
| | Uterine | | |
| Spill Potential | None | Yes | None |
| Individually | Yes | No | No |
| tailored | | | |
| radiation fields | | | |
| for each patient | | | |
| Radiation | + | ++++ | +++++ |
| damage to | | | |
| normal tissue | | | |

+to +++++ indicates the lowest (+) to highest (+++++) likelihood of radiation damage to normal tissue

As shown, in addition to the need to improve the delivery of radiation therapy within the human body to treat diseases such as cancer, is the need to improve the method of depositing radioactive materials on substrates that are used in radiation therapy.

A variety of methods are traditionally used to form metal coatings on substrates. These include electrodeposition and electroless deposition. Electrodeposition depends on the use of applied voltage to produce metal deposition, while electroless deposition depends on chemical reactions (including, the chemical reduction of a metal) independent of applied voltage. See, e.g., Dini, J. W., Developments and Trends in Electrodeposition, SAMPE Quarterly (1989) 28-32; and Ohno, I. Electrochemistry of Electroless Plating, Materials Science and Engineering, Vol. A146 (1991) 33-49.

A wide variety of solutions for electrodeposition and electroless deposition are known, as theoretically any element or combination of elements, including metals and non-metals, can be added to a carrier metal to provide a suitable coating solution, wherein the carrier metal is present as an ion. In particular, metalloids including phosphorus and boron can be added to a carrier metal to provide a coating solution. Commonly used carrier metals include nickel, copper, cobalt, platinum, palladium, chromium, gold and silver. Particularly common are nickel and nickel alloy coating solutions, including nickel-phosphorus, nickel-boron, palladium-nickel, nickel-chromium, nickel-cobalt, nickel-phosphorus-boron, and copper-nickel chromium. Solutions are typically aqueous.

Electroless coatings are significantly more uniformly deposited than electrodeposited coatings, and are particularly desirable for coating complex shapes, including tubes and large components. Electroless deposition of nickel-phosphorus coatings, in particular, is well known. In general, electroless nickel phosphorus (ENP) coatings are dense, non-porous metal glass structures resembling polished stainless steel. ENP coatings typically contain between 3 and 13% by weight phosphorus, with the percentage significantly influencing both the chemical and physical properties of the coating. High phosphorus ENP coatings provide superior corrosion protection and are generally more continuous that lower phosphorus ENP coatings. R. P. Tracey, Practical Guide to Using N-P Electroless Nickel Coatings, Materials Selection and Design, 1990. ENP coatings are generally highly adhesive, providing resistance to chipping and peeling under extreme conditions. Electroless coatings may be amorphous or crystalline in structure.

Materials to be coated by electroless deposition are commonly metal. Electroless coatings can be applied to most metals and alloys, including steel and stainless steel, iron, aluminum, titanium, magnesium, copper, brass, bronze and nickel. In some cases, in addition to cleaning and removing surface oxides, the metal or alloy must be pre-treated to provide a catalytic surface for the electroless coating. For example, for coating Elgiloy™ with ENP, the surface must be coated (i.e., by electrodeposition or electroless deposition) with Ni prior to being coated with ENP. Electroless deposition may also be used to coat a variety of materials that are generally non-conductive, including plastics, glasses and ceramics, and composite materials. Coating of polymers generally requires additional steps to activate the polymer surfaces. A variety of processes are known for making polymer surfaces catalytic to the coating process. A tin-palladium catalyst, for example, can be absorbed onto the surface of the substrate, or applied as a catalytic coating.

Electroless deposition is carried out by immersing the substrate to be coated in an coating solution or bath comprising a carrier metal ion and a reducing agent. In ENP coating solutions, the most common reducing agent is hypophosphite ion (H₂PO₂). (Tracey, 1990). The metal ions are chemically reduced in the presence of the reducing agent and deposited onto the substrate surface. Deposition rates are typically 10-20 microns per hour. Typical commercial ENP coating are from about 2.5 to about 125 microns thick. Id. Thicker coatings are typically required for rough surfaces.

Metal coatings may also be formed by electrodeposition. For example, nickel-phosphorous coatings may be produced by electrodeposition, and have comparable properties to those prepared via electroless deposition. Weil et al., Comparison of Some Mechanical and Corrosion Properties of Electroless and Electroplated Nickel-Phosphorous Alloys, Plating and Surface Finishing (February 1989) 62-66.

Materials to be coated by electrodeposition include most metals and alloys, which in some cases must be clean and oxide free to provide a catalytic surface for electrodeposition. In certain circumstances, polymers may also be coated by electrodeposition. For example, plastics incorporating conductive particles can be coated by electrodeposition. Intrinsically conductive polymers may also be coated by electrodeposition. Generally, electrodeposition rates of Ni—P are higher than normally obtained via electroless methods. Also, electroplating solutions are more stable and have fewer replenishment problems. However, electrodeposited Ni—P does not coat complicated shapes with as uniform a thickness as ENP.

Electrodeposition is carried out by immersing the substrate to be coated in a coating solution or bath comprising a carrier metal ion and a radioisotope. Unlike electroless deposition, electrodeposition requires an applied current. In general, a reducing agent such as is necessary for electroless deposition is not required for electrodeposition, although reducing agents are not uncommonly present for electrodeposited Ni—P coatings, for example.

Methods for producing radioactive metal articles are also known. For example, it is known to manufacture a metal article comprising a radioisotope, e.g., by alloying the radioisotope with a metal or alloy or by ion implantation with a radioactive element. It is also known to manufacture non-radioactive metal articles which are subsequently made radioactive, e.g., by neutron bombardment. Each method of preparing radioactive metal articles, however, is associated with particular disadvantages. Manufacture of alloys using radioactive elements, for example, is problematic because many of the most desirable radioisotopes (e.g., P) show limited solubility as equilibrium alloying ingredients. Moreover, health physics safety issues associated with the manufacture of various articles effectively prohibit certain methods of manufacture.

The use of neutron bombardment to produce radioactive metal articles is similarly problematic, given limited access to nuclear reactors and tremendous costs. Neutron bombardment also constrains the size of components which can be irradiated. Moreover, neutron bombardment activates all components of the metal article that are susceptible to neutron activation, so that undesirable and potentially dangerous radioisotopes may be generated. Many standard alloy components, including Fe and Cr, form undesirable radiation reaction products. Thus, metals and alloys subject to neutron bombardment must be extremely pure and free of problematic elements, e.g., Na.

The use of radiation is known to be effective in killing cancerous cells and preventing their re-growth. Coupled with the need to improve the method of depositing radioactive materials described above is the need to improve the delivery of radiation therapy within the human body to treat diseases, such as cancer.

The present invention is a device that enables accurate and safe delivery of radiation locally for the treatment and prevention of cancer and other proliferative disorders. The invention takes advantage of the ability to selectively deposit radioactive isotopes onto metallic and other surfaces, such as rods, needles, wires or expandable devices, in a well-controlled manner. In addition, the concentrations of these isotopes, and therefore the radiation fields, can vary along the length and width of the of the rod. As a result, a variation or gradient in the radiation field can be obtained. The use of the coating techniques described above, e.g., electroplating, electroless deposition, polymer coating, ceramic coating, composite coating, allows for the accurate deposition of gamma and beta isotopes onto metal and other substances. The isotopes can be chosen for their ability to emit alpha particles, beta particles, gamma rays and/or x-rays. A variation or gradient in the radiation field provides a distinct advantage to effectively treating tumors, especially when used in combination with tumor mapping. Computer programs, such as those used in computer assisted tomography (CAT) scans and magnetic resonance imaging (MRI) techniques allow an accurate three-dimensional image of body structures, including tumors, to be obtained. The permanent placement of radiation sources can be used to design radiation fields that have a morphology that is substantially identical to the tumor. This allows one to design a radiation field that not only minimizes the amount of normal tissue that will be irradiated, but which maximizes the amount of malignant tissue that can be irradiated. The exclusion of a significant amount of normal tissue enables the use of radiation doses that are much higher since the major limitation with current systems is radiation toxicity to normal tissue. Since higher radiation doses can be used, the number of patients cured with this treatment should be higher than with current therapy.

As a result, this technology promises to increase the survival rate from cancer, decrease the major side effect associated with radiation therapy, decrease patient inconvenience and drastically decrease health care expenditures. This technology can be applied to cancers involving breast, brain and prostate. With minor modifications, other cancers can also be effectively treated with this technology. It is also feasible that non-malignant, proliferative disorders can also be treated with this technology.

It is one object of the present invention to provide radioactive coated substrates.

It is a further object of the present invention to provide substrates coated with multiple layers of radioactive coatings.

It is yet a further object of the present invention to provide a medical device, or a component of a medical device, coated with one or more radioactive coating layers.

It is still a further object of the present invention to provide a method of making a substrate having a variable radioactive coating or coatings capable of producing an asymmetric radiation field.

It is yet a further object of the present invention to provide a substrate having a variable radioactive coating or coatings capable of producing an asymmetric radioactive field.

It is an object of the present invention to provide a brachytherapy device coated with a variable radioactive coating or coatings capable of producing an asymmetric radioactive field.

It is a further object of the present invention to provide a method of producing a radiation field corresponding to a target field.

It is a still further object of the present invention to provide a method of producing a radiation field corresponding to the morphology of a tumor.

It is another object of the present invention to provide a method for treating cancerous tumors or non-malignant proliferative disorders using a device having a radioactive coating.

### SUMMARY OF THE INVENTION

The present invention relates to radioactive coating solutions, radioactive sols and sol-gels, methods used to form a radioactive coatings on a substrate, and to radioactive coated substrates, particularly medical devices. It is known that radiation therapy can reduce the proliferation of rapidly growing cells, including cancer cells. The present invention utilizes a radioisotope source with a device for securely placing a radiation source in or around a tumor. More specifically, the present invention is directed to a brachytherapy device comprising a radiation source that is advanced into a tumor and anchored to keep the radiation source stationary. Since the radioisotope source is stationary, the radiation source not only predictably inhibits the growth of hyperproliferating cells, but it reduces the damage to healthy tissue adjacent the tumor.

The present invention relates to a device that can be advanced into a tumor, an organ with a tumor, or a cavity from which a tumor can be removed. The device comprising an outer housing and a central portion located within the outer housing, wherein the outer housing can have an opening on at least one end. The central portion comprising an anchor with a cable securely fixed to the anchor. The device further comprises a separate clip which helps maintain the central portion in a fixed position by attaching, while in tension, the part of the central portion coming through the skin to the skin.

The present invention relates to radioactively coated substrates. Suitable substrates include, but are not limited to, metals, alloys, polymers, plastics, ceramics and composites. In a particular embodiment of the present invention, the substrate is a medical device formed from such materials, or a component thereof. Representative medical devices include catheters, guidewires, stents, and brachytherapy devices. More particularly, the substrate is a needle, a rod, a wire or a clip used in a brachytherapy device.

The present invention also relates to a method of making a substrate having a radioactive coating, optionally a variable radioactive coating capable of producing an asymmetric radiation field, as well as to substrates having a radioactive coating. In a particular embodiment, the present invention relates to a substrate used in a brachytherapy device, as defined above, having a variable radioactive coating capable of producing an asymmetric radiation field.

The present invention also relates to a method of producing a radiation field corresponding to a target field. In particular, the method of the present invention involves the design or selection of a brachytherapy device having a variable radioactive coating capable of producing an asymmetric radiation field, which can be used alone or in combination with other such devices to produce a radiation field that closely corresponds to the morphology of a tumor.

The present invention advantageously permits production of radioactive substrates by virtue of a radioactive coating or coatings applied thereto. The present invention overcomes limitations of the traditional alloying and nuclear bombardment methods used to render metal articles radioactive to provide a radioactive metal coating which can be formed from a wide array of radioisotopes, including insoluble radioisotopes, relatively safely and inexpensively.

In certain other embodiments, the present invention advantageously permits production of a radioactive coated brachytherapy device which can be used alone or in combination with other such devices to produce a radiation field closely corresponding to the morphology of a tumor, thus reducing radiation damage to adjacent normal tissue. Accordingly, the dose of radiation that can be given to a tumor is increased with less damage to the adjacent normal tissue than is seen with radiation doses currently in use. This will most likely result in a higher response rate of tumors to brachytherapy, and in many cases, higher patient survival rates.

Thus, the present invention also relates to a method of treating cancer using a brachytherapy device according to the claimed invention. The advantages of the present invention in the treatment of cancer are best illustrated by comparing the standard treatment of breast cancer with a method according to the claimed invention.

Using the standard of care as it is practiced today, a woman with a newly diagnosed, well-localized breast tumor with negative axillary lymph nodes would usually be treated with removal of the tumor, followed by radiation therapy beginning several weeks after surgery and requiring at least 6 weeks to complete. Not uncommonly, the patient would later undergo yet another surgical procedure for breast reconstruction. The total surgical and radiation therapy treatment commonly consists of two surgeries and at least 6 weeks of daily (5 visits/wk) radiation therapy visits. The patient would then have to complete a course of chemotherapy.

With the present brachytherapy method, the patient undergoes tumor removal and implantation of the inventive device at the same procedure. The inventive device is a breast implant modified with openings or ribs on its outer layer. The patient then is discharged. After recovering, the patient returns for an outpatient CT scan of the chest. Since the inventive device has radio-opaque markers, the position of the device in the breast can be accurately determined. The radiation oncologist and physicist then evaluate the position of the device and decide on the optimal radiation field, the regional intensity of the field, the number of sources to be used and the desired placement of the sources. This information, along with the CT images, is used to custom make a brachytherapy device.

Wires are specially made at an outside facility to create the desired radiation field and loaded into an insertion/removal tool. This wire-loaded tool is then sent to the radiation oncologist. At the patient's first outpatient visit the wires are implanted onto the surface of the breast implant in a single step using the insertion/removal tool. After the prescribed period (several days-weeks) of radiation exposure, the patient returns for her second outpatient visit, the wires are removed and the neck of the implant is disconnected from the device. The patient is finished with radiation therapy. Because the implant was placed at the time of tumor removal, there is no need for a surgical procedure for breast reconstruction. The total surgical and radiation therapy treatment consists of one operation (tumor removal and device implantation), one outpatient CT scan and two office visits.

Another embodiment of a brachytherapy device for the treatment of breast cancer has an outer layer composed of a drug-delivery polymer that is loaded with an anti-metabolite (e.g., paclitaxel) that can locally deliver drug in a predictable manner, which should at least decrease (if not eliminate) the amount of systemic drug necessary for the course of chemotherapy that usually follows radiation therapy. This would lead to a significant decrease in chemotherapy-associated side effects and provide higher drug levels at the target site than can be obtained with systemic therapy.

Advantages of the claimed breast cancer device include decreased radiation toxicity. That is, the ability to tailor the radiation field to a particular patient should markedly decrease the amount of normal tissue adjacent to the desired radiation field that is susceptible to radiation injury. Preferably, the radiation field extends not more than 3 cm, more preferably not more than 2 cm, into the normal tissue adjacent the excision cavity formed by removal of the tumor.

In addition, the breast cancer device provides the potential for higher local radiation doses. The accurately tailored radiation fields will, by definition, minimize the amount of normal tissue within the radiation field. Since there is less danger of radiation injury, it is conceivable that higher radiation doses can be employed, which could potentially lead to higher cure rates.

Convenience is another benefit of the claimed breast cancer device. This device is implanted at the time of tumor removal and requires only one outpatient CT scan and two office visits. This is a marked improvement of the two operations (tumor removal, breast reconstruction) and daily radiation therapy sessions for 6 weeks. Additionally, treatment with the breast cancer device is particularly suited to patients who live a significant distance from a radiation treatment facility. All that is necessary for this device are the professional staff (surgeon, radiation oncologist, radiation physicist), and appropriate safety measures.

Since this device is a modified breast implant, it eliminates the need for a separate breast reconstruction procedure. The number of outpatient visits for radiation therapy will be decreased from approximately 30 to two, providing a significant cost savings in the treatment of breast cancer, for example.

Many patients with cancer, especially solid tumors involving the breast and brain, undergo surgery to remove the tumor, followed by chemotherapy and radiation therapy. The goal of the chemotherapy is to kill the tumor cells. Most chemotherapy agents work by inhibiting the production of DNA. Since these agents are not specifically targeted to the tumor cells, they affect all of the cells in the body that are synthesizing DNA. Therefore, the regions that suffer the most toxicity from chemotherapy agents are those that high rates of DNA synthesis. Bone marrow toxicity (inability to make red blood cells and platelets), infections (due to inability to make white blood cells), hair loss and GI toxicity (due to the inability to make cells in the gut) are all related to the intended action of the chemotherapeutic agent. Since the target cannot be specified, the normal cells that have high turnover (and DNA synthesis) rates are killed along with the abnormal or cancerous cells.

In general, doses of many chemotherapeutic agents are limited by their toxic effects on the normal body cells. If a method was developed that could maximize drug delivery to the abnormal cells and minimize the drug levels in normal cells, more drug could be administered, which could increase cure rates.

If an implant could be placed at the site of tumor removal and that implant could release drugs locally, the target site (immediately around the tumor cavity) would receive very high amounts of drug, while the rest of the body would receive a much smaller amount. One solution to the above problems involves the use of a breast implant that would be placed after tumor removal. The implant could deliver drugs in either of two ways:

(1) The outer layer of the implant could be composed of a drug delivery polymer (such as a hydrogel). Before the implant is placed in the body the polymer could be loaded with drug.

(2) The outer layer could be composed of a porous layer. The implant could have a removable neck that is left has a lumen that connects to the outer porous layer. Drug could be infused into the lumen in the neck of the device and into the porous layer.

It is understood that the drug deliver system described above could be used alone, or in addition to radiation therapy using the previously described brachytherapy device, to provide a combination of radiation and chemotherapy, when treating cancer, for example.

These and other advantages of the present invention will be apparent to those skilled in the art in view of the disclosure set forth below.

### BRIEF DESCRIPTION OF THE FIGURES

FIG. 1 is an isodensity curve of the radioactive coating applied to a catheter according to Example 1, as measured along the catheter's long axis, illustrating uniformity of deposition.

FIG. 2 is an isodensity curve of the radioactive coating applied to a catheter according to Example 1, as measured along the catheter's short axis, illustrating uniformity of deposition.

FIG. 3 is a Ni-25 atomic %P electroless coating deposited onto Elgiloy™ in sheet form, viewed in cross-section via scanning electron microscopy (SEM).

FIG. 4 is a coated and uncoated catheter component by SEM images. 4A depicts a device coated with a Ni-26 atomic %P electroless coating. The coating is approximately 7 microns thick, and is uniform in appearance. 4B depicts an uncoated device.

FIG. 5 is a cross-section of the device of FIG. 4A. 5A depicts SEM at 100×. 5B depicts SEM at 300×.

FIG. 6 is an energy dispersive x-ray spectrum from a Ni—P electroless electroless coating, showing Ni and P peaks, corresponding quantitative analysis indicates concentration of coating being, about 26 mol or atomic %P (or about 15.8 wt. % P).

FIG. 7 is an x-ray diffraction spectrum the uncoated Elgiloy™ and the Ni—P electrolessly coated Elgiloy™ of FIG. 4. The uncoated alloy shows crystalline peaks consistent with the substrate; the coated alloy shows a diffuse peak consistent with the coating being amorphous as expected for a high phosphorus coating.

FIG. 8 represents substrates having a radioactive coating or coatings formed thereon.

FIG. 9 represents the use of a brachytherapy to treat a tumor with local radiation.

FIG. 10 is a brachytherapy device, including (a) a complete device, which includes: (b) a central portion; (c) housing (front view); (d) housing (side view); and (e) a clip.

FIG. 11 is a brachytherapy device, including (a) a complete device, which includes: (b) a top portion (side view); (c) a top portion (bottom view); (d) a central portion; (e) housing; and (f) a clip.

FIG. 12 is a brachytherapy device, including an expandable device containing ribs for radioactive rods.

FIG. 13 represents a breast having an excision cavity created by the removal of a tumor.

FIG. 14 is the breast of FIG. 13, wherein the cavity is filled with the brachytherapy device of FIG. 12.

FIG. 15 represents a shielded insertion/removal tool that also stores the radioactive substrates.

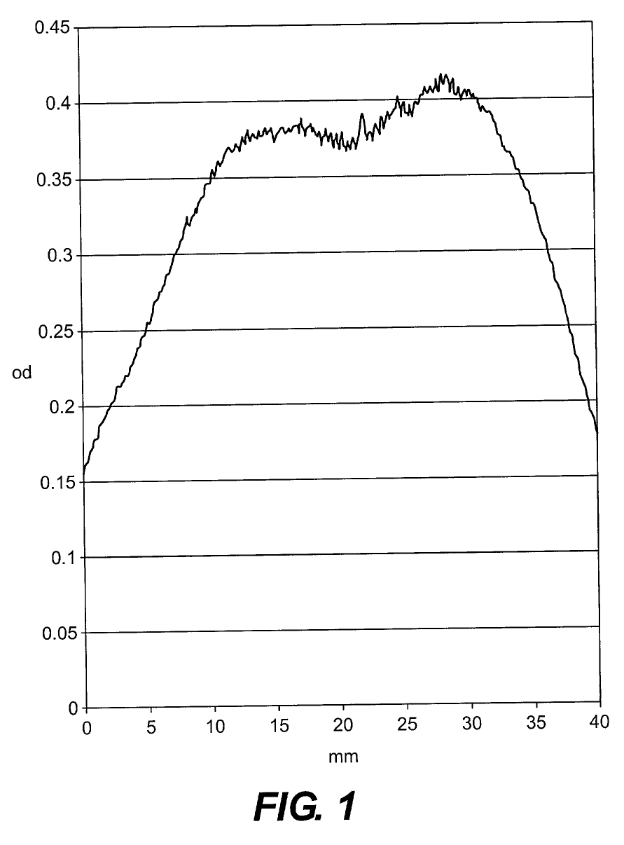

### DETAILED DESCRIPTION OF THE INVENTION

The invention disclosed herein relates to device used to treat tumors, wherein the device comprises a substrate having at least one radioactive coating. A “substrate” for purposes of this invention is defined as an object having a surface which is able to have a radioactive coating applied thereto. A substrate can be placed within or adjacent to at least a portion of a desired target to be treated, such as, for example, a tumor, in a manner which allows it to remain in a substantially fixed position for an extended period of time. During this period, the patient may have a fair degree of mobility to go about his or her every day activities substantially uninhibited.

An “anchor” for purposes of this invention is an object which can be placed within or adjacent to at least a portion of a desired target to be treated, such as, for example, a tumor. An anchor contains at least one end that maintains a rigid point of support within the body such that it allows the substrate, as defined above, to remain in a substantially fixed position for an extended period of time. An anchor may take any shape, with preferred embodiments being flat (or planar), conical or a shape typically used in the nautical art, e.g., stockless, mushroom or admiralty.

A “clip” for purposes of this invention is an object for holding a substrate, as defined above, to the skin or mucosa. A clip includes any type of clasp or fastener that can be attached to the skin or mucosa, and that maintains tension on a substrate such that the substrate remains in a substantially fixed position for an extended period of time.

The term “adjacent” for purposes of this invention means “close to” or “in proximity with” and may include direct physical contact.

A device according to the present invention is a brachytherapy device comprising a substrate that includes at least one radioactive coating layer formed thereon. The radioactive coating layer having a total radioactivity that varies in at least one dimension of the device. While not limited to any certain form or shape, the substrate can be a hollow needle, a solid needle, or a more flexible substrate, such as a wire or cable.

As previously explained, the radioactive coating layer may comprise a variable concentration of a radioisotope along a given dimension, and may include one or more additional radioactive coating layers covering at least a portion thereof. The radioactive coating layer can be different from the first layer to the additional radioactive coating layers. For example, the first radioactive coating layer may comprise ¹⁹²Ir and the additional radioactive coating layer or layers may comprise a radioisotope selected from the group consisting of ³²P, ¹⁰³Pd and ¹⁹⁸Au. It is possible that the first radioactive coating layer and the additional layers may comprise a variable concentration of radioisotope along said dimension.

In addition, the radioactive coating may comprise a metal matrix and a radioactive dispersed phase. It is also possible that a brachytherapy device according to the present invention can comprise a catalytic coating layer interposed between the substrate and the first radioactive coating layer. In fact, it is possible that one or more catalytic coating layers may be interposed between one or more additional radioactive coating layers.

In one embodiment, a brachytherapy device according to the present invention may comprise a substrate as described above, further including an anchor attached to the substrate at one end and a clip attached to said substrate at the end opposite the anchor. The clip, which is fastened to the skin, maintains tension in the coated wire or substrate, and thus ensures that the coated substrate remains securely anchored and thus stationary in the body. This leads to a predictable radiation dose.

The anchor can have either a flat shape or a conical shape with two side flaps that can attach to human tissue or a tumor. When the anchor is conical in shape, the inside of the cone may include a threaded portion, which may be used to securely attach the anchor to the central portion. Of course, this would require the central portion to have a threaded portion as well, which is within the scope of this invention.

The present invention is also directed to a method for treating a tumor with radiation by using a brachytherapy device described above. For example, such a method may comprise advancing a brachytherapy device through the skin or mucosa and into a tumor. In this embodiment, the brachytherapy device may comprise a central portion, which comprises a wire or cable and an anchor securely attached to the wire or cable, wherein the wire or cable contains a radioactive segment, i.e., it includes at least one radioactive layer. The central portion is located within a housing, which may contain an opening at one end.

After the complete brachytherapy device is advanced to the desired position, the housing is withdrawn such that the wire or cable is left in the body. Tension is next put on the wire or cable by pulling the wire or cable, which deploys the anchor such that the anchor is securely fixed within the body. To maintain the radioactive coated substrate in the desired location, a clip is attached on the wire or cable coming through the body. The clip is then attached to the skin or mucosa while keeping the wire or cable in tension.

In another embodiment of the present invention, the brachytherapy device comprises rods having at least one radioactive coating layer formed thereon and an expandable or inflatable device comprising openings or ribs in which the rods are placed. An expandable or inflatable device encompasses any device traditionally used as a surgical implant, such as devices used as breast implants.

The terms “openings” or “ribs” are defined as any entrance which allows rods to be placed adjacent to the expandable or inflatable device and which also securely hold the rods in such a position for an extended period of time, which is defined as anywhere from a few hours to a few months. The openings or ribs are interspersed at defined intervals on the expandable or inflatable device.

Optionally, the radioactive coating layer on the rods has a total radioactivity that varies in at least one dimension of the device. This type of device is suited for treating the area around the location from which a tumor was surgically removed from the body, i.e., the excision cavity.

This method comprises surgically removing a tumor from a human body thereby forming an excision cavity in the human body. Placing a brachytherapy device in the cavity, wherein the brachytherapy device comprises rods having at least one radioactive coating layer formed thereon, wherein the rods are placed into the openings or ribs in the inflatable device.

The present invention also relates to a radioactive coating solution comprising at least one carrier metal ion and a radioisotope. In a particular embodiment, the radioactive coating solution comprises a carrier metal ion, a radioisotope and a reducing agent. Suitable carrier metals ions include, without limitation, nickel, copper, cobalt, palladium, platinum, chromium, gold and silver ions. In one embodiment of the present invention, the carrier metal ion is nickel ion. The concentration of carrier metal ion in the radioactive coating solution may vary, as would be understood by one skilled in the art. A representative carrier metal ion concentration is from about 1 to about 30 g/L. Carrier metal ion concentrations from about 3 to about 15 g/L are particularly suitable for use with radioactive coating solutions wherein the carrier metal ion is nickel.

Radioisotopes suitable for use in the coating solution of the present invention include beta, gamma, or alpha emitters. In a particular embodiment, the radioisotope is a non-metal (e.g., ³²P). Beta radiation penetrates only a limited distance through human tissue, and is therefore particularly desirable for localized radiation therapy. Beta emitters suitable for use in the present invention include, but are not limited to, ¹⁴C, ³⁵S, ⁴⁵Ca, ⁹⁰Sr, ⁸⁹Sr, ³²P, ³³P, ³H, ⁷⁷As, ¹¹¹Ag, ⁶⁷Cu, ¹⁶⁶Ho, ¹⁹⁹Au, ¹⁹⁸Au, ⁹⁰Y, ¹²¹Sn, ¹⁴⁸Pm, ¹⁴⁹Pm, ¹⁷⁶Lu, ¹⁷Lu, ¹⁰⁶Rh, ⁴⁷Sc, ¹⁰⁵Rh, ¹³¹I, ¹⁴⁹Sm, ¹⁵³Sm, ¹⁵⁶Sm, ¹⁸⁶Rc, ¹⁸⁸Rc, ¹⁰⁹Pd, ¹⁶⁵Dy, ¹⁴²Pr, ¹⁴³Pr, ¹⁴⁴Pr, ¹⁵⁹Gd, ¹⁵³Gd, ¹⁷⁵Yb, ¹⁶⁹Er, ⁵¹Cr, ¹⁴¹Ce, ¹⁴⁷Nd, ¹⁵²Eu, ¹⁵⁷Tb, ¹⁷⁰Tm, and ¹⁹⁴Ir.

Gamma emitters suitable for use in the present invention include, but are not limited to, the group comprising ¹³⁷Cs, ⁶⁰Co and ¹⁹²Ir. Similarly, suitable alpha emitters include, but are not limited to, the group comprising ²²⁶Ra and ²²²Rn. Other radioisotopes suitable for use in the present invention include, but are not limited to, ¹²⁵I, ¹⁹²Ir and ¹⁰³Pd.

In a particular embodiment, the radioisotope is ³²P, ¹⁰³Pd or ¹⁹⁸Au. Representative properties of these isotopes are given in Table 2.

TABLE 2
Properties of ¹⁹⁸Au, ³²P and ¹⁰³Pd

| | | | | | Exposure | Specific | | |
|---|---|---|---|---|---|---|---|---|
| | | | | | Rate | Rate | | |
| | | | | | Constant | Constant | | HVL |
| Element | Isotope | E_{b} | E_{g} | [symbol] _{g} | Rm²/h/mCi | Rm²/h/mCi | T_{1/2} | (water; cm) |
| Gold | ¹⁹⁸Au | 0.96 | 0.412-1.09 | 0.4 | 2.376 | 2.327 | 2.7 d | 7.0 |
| | | | | 1 6 | (0.0773) | (0.0549) | | |
| Palladium | ¹⁰³Pd | — | 0.02-0.48 | 0.0 | 1.48 | | 17 d | 1.6 |
| | | | | 2 1 | (0.0331) | | | |
| Phosphorus | ³²P | 1.71 | None | — | | | 14.3 d | 0.1 |

The above isotopes are preferred since they are not only familiar to the radiation oncology community (¹⁹⁸Au and ¹⁰³Pd have been used in prostate brachytherapy, while ³²P is a common isotope in coronary brachytherapy), but the non-radioactive forms of all three elements have been used in the field of electrodeposition for decades. This is one of the methods used for differential coating of the wires according to some embodiments of the present invention. Therefore, the methods for coating metal surfaces are well known and expertise in the field is widely available.

In a particular embodiment, the coating solution of the present invention is prepared by adding a water-soluble phosphorus compound to the coating solution, wherein at least a fraction of the P is ³²P Put another way, ³²P is present in the coating solution as an aqueous solution of phosphorous-containing ions. In a particular embodiment, the source of ³²P is any compound containing hypophosphite (H₂PO₂). Non-limiting examples of hypophosphite compounds suitable for use in the present invention include hypophosphorus acid, sodium hypophosphite, ammonium hypophosphite, potassium hypophosphite and lithium hypophosphite. In a particular embodiment, aqueous NaH₂PO₂.H₂O or NaH₂PO₄.2H₂O is added to the coating solution, wherein at least a fraction of the P is in the form of ³²P.

In a further embodiment of the present invention, the source of ³²P is any compound containing phosphite (HPO₃²⁻). Phosphorous acid, H₃PO₃, provides a non-limiting example of a phosphite material suitable for use in the present invention. In a still further embodiment of the present invention, the source of ³²P is any compound containing orthophosphate (PO₄³⁻). Orthophosphoric acid, H₃PO₄, provides a non-limiting example of an orthophosphate compound suitable for use in the present invention.

The amount of radioisotope present in the radioactive coating solution may vary, as would be understood by one skilled in the art. A representative specific activity is from about 0.1 to about 5000 Ci/g, and more particularly, about 20 Ci/g (or 64/Ci/mole) which amount is particularly suitable for coating solutions comprising ³²P in the form of hypophosphite or hypophosphorus acid. This representative specific activity falls below the theoretical maximum for ³²P (i.e., slightly greater than 9000 Ci/mol, or 9,000,000 Ci/mol). This representative amount is particularly suitable where NaH₂PO₂.H₂O is the only reductant present in an electroless Ni—P coating solution.

Suitable reducing agents for use in the coating solution of the present invention include, but are not limited to, hypophosphites, formaldehyde, borohydride, dialkylamine boranes (e.g., dimethyl borane), and hydrazine. Each of these reductants has a particular condition range that is well known to one skilled in the art. In particular, for ENP, NaH₂PO₂ is commonly used as a reductant, with a representative range from about 5 g/l to about 50 g/l.

As would be evident to one skilled in the art, the radioisotope of the coating solution may be the radioactive form of an element present as the reducing agent, or a component thereof. For example, in a given coating solution, the radioisotope may be ³²P while the reducing agent might be NaH₂PO₂. Alternatively, the radioisotope may be the radioactive form of the carrier metal. For example, in a given coating solution, the radioisotope may be ¹⁹⁸Au, while the carrier metal is also Au.

In a particular embodiment of the present invention, the coating solution comprises NiSO₄ (26 g/l), NaH₂PO₂.H₂O (26 g/l), Na-acetate (34 g/l), lactic acid (18 g/l) and malic acid (21 g/l), wherein at least a fraction of the P is ³²P. In a further embodiment of the present invention, the coating solution comprises AuCN (2 g/L), NaH₂PO₂ (10 g/L), KCN (0.2 g/L), wherein at least a fraction of the P is ³²P. In a still further embodiment, the coating solution comprises AuCN (2 g/L), NaH₂PO₂ (10 g/L), KCN (0.2 g/L) wherein at least a fraction of the Au is ¹⁹⁸Au. In a still further embodiment, the coating solution comprises AuKCN (5.8 g/L), KCN (14 g/L), KOH (11.2 g/L), and KBH₄ (21.6 g/L), wherein at least a fraction of the Au is ¹⁹⁸Au.

Additional components may be added to the coating solution to vary the physical and chemical characteristics of the coating.

The present invention further relates to a method of forming a radioactive coating on a substrate, which coating comprises at least one carrier metal and a radioisotope. The coating is formed by contacting the substrate with a radioactive coating solution comprising a carrier metal ion and a radioisotope. The coating solution may have the properties described above, as one skilled in the art would appreciate. Various coating techniques known in the art are suitable for use in the present invention including, but not limited to, electroless deposition, electrodeposition, chemical vapor deposition, physical deposition, thermal spraying, sol-gel methods, or any combination thereof. Certain methods may be more suitable for certain substrates, as would be understood by one skilled in the art.

Substrates coated according to the present invention may include, but are not limited to, metals, alloys, polymers, plastics, ceramics and composites. As previously described, the substrate is a medical device, such as a catheter, guidewire, stent or brachytherapy device. A brachytherapy device includes an expandable or inflatable device, a rod, a needle (hollow or solid), a cable or wire, a clip, or a component thereof.

Coating Techniques

1. Electroless Deposition

In a particular embodiment of the method, electroless deposition is used to form a radioactive coating on a substrate. In this embodiment, the substrate is contacted with the radioactive coating solution, for a time, at a concentration, a temperature and pH sufficient to chemically deposit a radioactive metal coating on the substrate. It may be necessary to clean the substrate and to remove surface oxides therefrom prior to deposition of the radioactive coating. It may further be necessary to coat the substrate with a catalytic coating or activating layer prior to electroless deposition of the radioactive coating, as would be recognized by one skilled in the art. The catalytic coating may be a non-radioactive Ni coating, for example. Suitable electroless coating solutions include, without limitation, electroless nickel coating solutions comprising hypophosphite, wherein at least a fraction of the P in hypophosphite is ³²P. Typical electroless nickel coating solutions are reviewed in W. Ying and R. Bank, Metal Finishing (December 1987), pp. 23-31, and in W. Riedel, Electroless Nickel Plating, ASM International (1991), pp. 9-32, which are incorporated herein by reference. Suitable electroless coating solutions also include electroless gold coating solutions comprising hypophosphite, wherein at least a fraction of the P in the hypophosphite is ³²P, as well as electroless gold solutions wherein at least a fraction of the Au is present as ¹⁹⁸Au. In a particular embodiment of the method, the radioisotope is the radioactive form of an element present as the reducing agent, or a component thereof (e.g., the radioisotope is ³²P, and the reducing agent is Na₂H₂PO₂). In a further embodiment of the method, the radioisotope is the radioactive form of the carrier metal (e.g., the radioisotope is ¹⁹⁸Au, while the carrier metal is Au).

Conditions for electroless deposition of a particular coating solution can vary, as would be recognized by one skilled in the art. These conditions also vary depending on the desired coating composition. Representative condition ranges include: (1) a pH range of from about 4.5 to about 10.0, and more particularly 4.8; (2) a temperature range of from about 60 to about 100° C., and more particularly 88° C.; (3) a metal concentration range from about 3 to about 15 g/L; (4) a deposition rate range of from about 0.5 to about 257 mil/hour, and more particularly 10 mil/hour; (5) a bath loading range of from about 0.1 to about 1.0 square feet per gallon, and more particularly 0.6 square feet per gallon; and (6) one or more reductants, from about 5 to about 50 g/L. A representative deposition of 1 μM at 10 μM/hour would take 6 minutes. These representative ranges are particularly suitable for use in electroless deposition of an electroless nickel-phosphorus coating solution having Na₂PO₂-H₂O as a reductant, wherein at least a portion of P is ³²P. Other suitable conditions for electroless nickel-phosphorus deposition are reviewed in Hur et al, Microstructures and crystallization of electroless Ni—P deposits, Journal of Materials Science, Vol 25, (1990), 2573-2584, which is incorporated herein by reference. Accurate temperature and metal concentration control are important to achieve uniform deposition rates. Various coating thicknesses are achievable, as would be apparent to one skilled in the art. A representative coating thickness ranges from about 0.1 to about 20 μm, and typically about 1.0 to about 2.0 μm. Optionally, a sealing or protective layer may be formed, i.e., a non-radioactive Ni sealing layer.

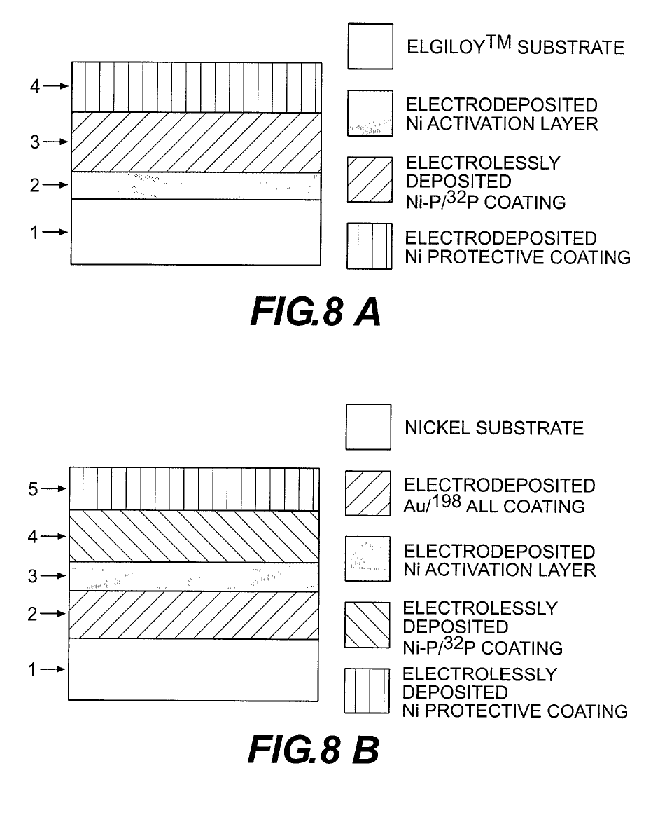

FIG. 8A depicts a substrate having an electroless radioactive coating. More particularly, this Figure depicts an Elgiloy substrate (1) coated by electrodeposition of a Ni activation layer (2), which activated substrate has a radioactive Ni—P/³²P layer (3) formed thereon. The radioactive coated substrate in FIG. 8 also has a Ni sealing layer electrodeposited thereon (4).

2. Electrochemical Deposition

The method of the present invention also includes the use of electrochemical deposition to apply a radioactive coating on a substrate. According to this method, the substrate is contacted with a radioactive coating solution for a time, at a concentration, at a temperature and voltage sufficient to electrically deposit a radioactive metal coating on the substrate. In some cases, it may be necessary to clean the substrate surface and to remove surface oxides prior to coating.

Electrochemical deposition of metals and alloys involves the reduction of metal ions from aqueous, organic and fused-salt electrolytes. This proposal will employ the deposition from aqueous solutions only. The reduction of metal ions M²⁺ in aqueous solution is represented by

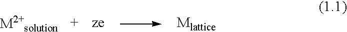

This can be accomplished via two different processes: (1) an electrodeposition process in which z electrons (e) are provided by an external power supply and (2) another, Electroless (autocatalytic) deposition process in which a reducing agent in the solution is the electron source (there is no external power supply involved). These two processes, electrodeposition and electroless deposition, constitute the process referred to as electrochemical deposition.

The basic components of an electrolytic cell for electrodeposition of metals from an aqueous solution are a power supply, two metal electrodes (M₁ and M₂), water containing the dissolved ions, and two metal/solution interfaces; M₁l/solution and M₂/solution. An electrolytic cell for electroless deposition includes only one electrode and no power supply. However, the solution is more complex. It contains water, a metal salt MA (M^{z+}; A^{z-}), and a reducing agent (Red) as the basic components The overall reactions of electrodeposition and electroless deposition may be used to compare these two processes. The process of electrodeposition of metal M is represented by

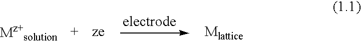

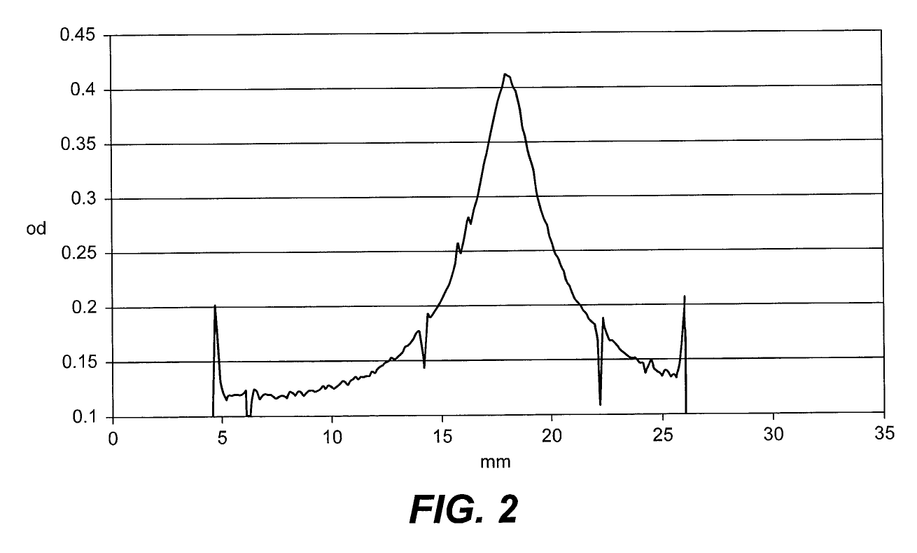

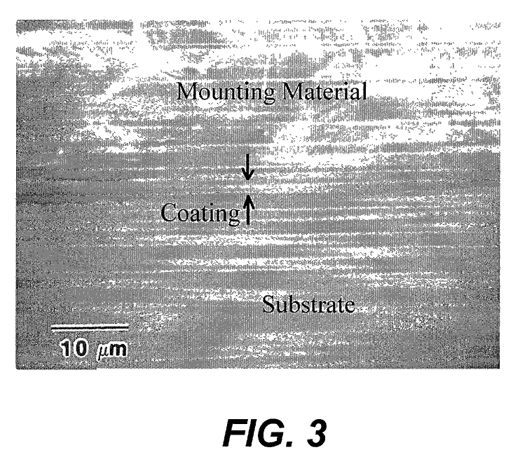

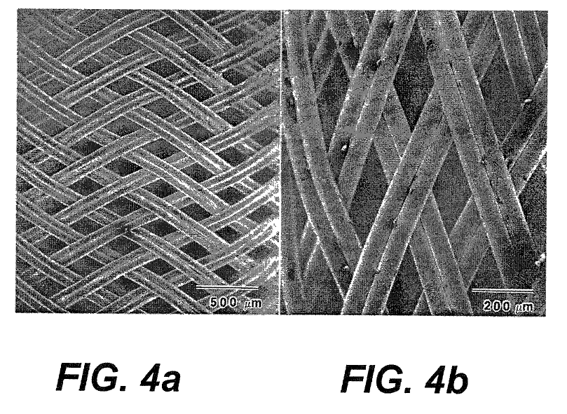

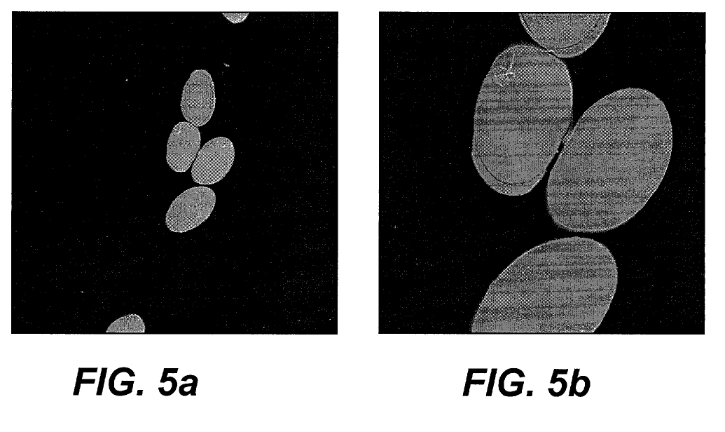

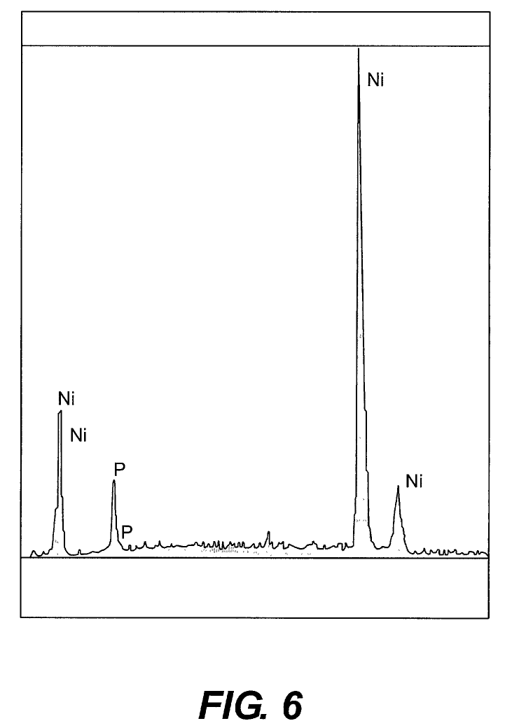

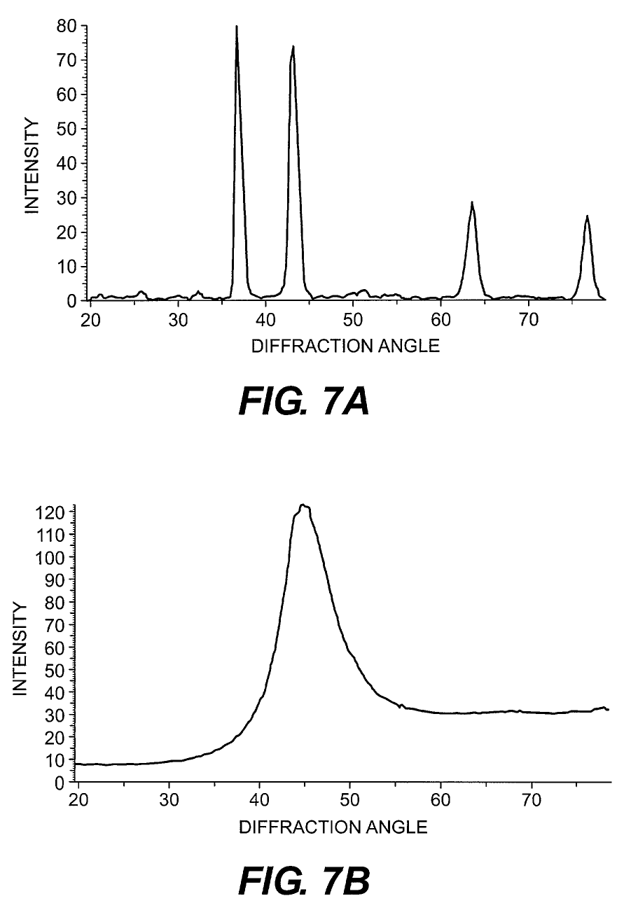

In this process z electrons are supplied by an external power supply (FIG. 2). The overall reaction of electroless metal deposition is

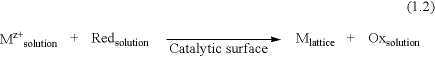

Where Ox is the oxidation product of the reducing agent Red. The catalytic surface may be the substrate S itself or catalytic nuclei of metal M dispersed on a noncatalytic substrate surface. In the electroless deposition process a reducing agent Red in the solution is the electron source; the electron-donating species Red gives electrons to the catalytic surface and metal ions M^{z+} at the surface. The reaction represented by Eq. (1.2) is conducted in such a way that a homogeneous reaction between M^{z+} and Red, in the bulk of the solution, is suppressed.

Unlike the electroless deposition technique described above, electrodeposition involves the use of two electrodes: a cathode and an anode. Here two separate electron-transfer reactions occur at two spatially separated electrode-electrolyte interfaces. At the cathode the reduction occurs (Eq. 1.1), and at the anode an oxidation reaction occurs, for example

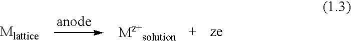

In the electroless deposition the two electrochemical reactions, reduction of M^{z+} solution and oxidation of Red solution occur at the same electrode, at one and the same electrode-electrolyte interface (Eq. 1.2). Thus, in the electroless deposition there is a statistical division of the catalytic sites on the substrate into anodic and cathodic sites. Since these catalytic sites are part of the same piece of metal (substrate), there is a flow of electrons between these sites.

In a particular embodiment of the method, the radioisotope present in the coating solution is a non-metal (e.g., ³²P) In a more particular embodiment, the coating solution comprises hypophosphite, phosphite, and/or orthophosphate, wherein at least a fraction of the P in the hypophosphite and/or the phosphite, and/or the orthophosphate is ³²P.

Suitable coating solutions for use an electrodeposition of radioactive metal coatings include, without limitation, a solution comprising nickel sulfate (150 g/L), nickel chloride (45 g/L), sodium hypophosphite (100 g/L), and orthophosphoric acid (50 g/L), wherein at least a portion of the P present is ³²P. Though conditions for electrodeposition vary, as would be familiar to those skilled in the art, representative conditions for electrodeposition of this radioactive coating solution include (1) a pH of about 6.0 to about 7.0; (2) a temperature of about 55-60° C.; (c) a current density from about 20 mA/cm² to about 500 mA/cm², and more particularly, about 80 mA/cm². In one embodiment, the method yields a dense, amorphous Ni—P coating, at a coating rate of 4.2 μm/h. Generally, for electrodeposited coatings, coating rates may vary considerably from, for example, about 0.1 to about 25 μm/hour using conventional electrodeposition. Various coating thicknesses are achievable, as would be apparent to one skilled in the art. A representative coating thickness ranges from about 0.1 to about 20 μM, and typically about 1.0 to about 2.0 μm. Optionally, a protective or sealing layer is formed onto the radioactive coated substrate, such as a non-radioactive Ni coating.

In a further embodiment, a radioactive coating solution suitable for electrodeposition comprises CrK(SO₄)₂.12H₂O (100 g/L), NiSO₄.6H₂O (50 g/L), (NH₄)₂SO₄ (50 g/L), NaH₂PO₂.H₂O (50 g/L), Na₃C₆H₅O₇.2H₂O (50 g/L), C₆H₈O₇ (25 g/L), H₃BO₃ (20 g/L), (NH₂)₂CS (0.01 g/L), C₁₀H₁₆O (0.333 g/L), and C₁₂H₂₅SO₄Na (0.1 g/L), wherein at least a fraction of the P in NaH₂PO₂.H₂O is present as ³²P. Representative conditions for electrodeposition of this solution include (1) pH from about 2 to about 4, and typically 2.3; (2) current density from about 5 to about 400 m A/cm2, and typically about 200 mA/cm2; (3) a temperature at or about room temperature.

In yet a further embodiment, a radioactive coating solution suitable for electrodeposition comprises NiSO₄6H₂O, NiCl₂6H₂O, NiCO₃, H₃PO₃, H₃PO₄ and saccharin, wherein at least a fraction of the P in H₃PO₃ is present as ³²P Representative conditions for electrodeposition of this solution include (1) pH from about 0.75 to 0.90 and typically 0.82; (2) a current density from about 5 to about 400 mA/cm², and typically about 20 mA/cm2; (3) a temperature between 40° C. and 95° C., typically 60° C.

3. Differential Coating

The ability to utilize electrodeposition for coating isotopes on metal allows the operator to closely control the thickness of the coating and thus the amount of radioactivity on the part by controlling the amount of isotope in the coating solution, the time of exposure to the current and the amount of current and voltage. For example, the thickness of a coating (and the radioactivity) can be doubled by doubling the duration of the electroplating process. Also, if a portion of the part was removed from the solution during the plating process, that portion will have a coating that is less thick (and thus less radioactive) than the rest of the part. In addition, several different isotopes can be placed on the same part, allowing for the specific design of radiation fields. This technology has the potential of revolutionizing the field of brachytherapy. The ability to specifically design radiation fields will minimize the amount of normal tissue within the radiation field. This should drastically decrease the radiation injury and allow for the use of higher doses of radiation and could result in higher cure rates.

The use of electrochemical deposition for differential coating of isotopes has the following advantages: (1) the amount of isotope deposition is directly proportional to the thickness of the coating can be easily controlled since it is directly proportional to the time spent in the bath; (2) electrochemical deposition can be used to place a coating on intact, completely assembled parts; (3) the process can be easily adapted for mass production; and (4) the cost of the process is reasonable.

The method of the present invention also includes the use of an applicator to apply a radioactive coating solution to the substrate. Suitable applicators include, but are not limited to, brushes and pens. Applicators for use in electroplating have an electrically conductive component. See U.S. Pat. No. 5,401,369 and U.S. Pat. No. 4,159,934, the entire contents of which are herein incorporated by reference.

In a particular embodiment of the present invention, the radioactive coating solution comprises at least one carrier metal ion and either an insoluble radioisotope or the insoluble compound of a radioisotope. In a particular embodiment, the radioactive solution also includes a reducing agent, with suitable reducing agents including identified above for radioactive coating solutions comprising at least one dissolved carrier metal ion and a dissolved radioisotope. The carrier metal ion is dissolved in solution, and may be, without limitation, nickel, copper, chromium, cobalt, platinum, palladium, gold or silver ion. In a particular embodiment, the carrier metal ion is copper, which can be dissolved in the coating solution in the form of any soluble copper salt, such as CuSO₄. In a further embodiment, the dissolved carrier metal ion is nickel.

The concentration of carrier metal ion in the radioactive coating solution may vary, as would be understood by one skilled in the art. A representative carrier metal ion concentration range would be from about 1 to about 30 g/L. Carrier metal concentrations from about 3 to about 15 g/L are particularly suitable for use with radioactive coating solutions wherein the carrier metal is nickel.

The insoluble radioisotope may comprise an insoluble radioisotope or insoluble compound of a radioisotope, such as an insoluble metal salt or oxide. Insoluble radioisotopes suitable for use in the coating solution of the present invention include, without limitation, insoluble ³²P, ⁹⁰Y and ¹⁹⁸Au. Insoluble compounds of radioisotopes include, without limitation, FeP, NiP, CoP, SnP, Ti₄P₃ and Y₂O₃, wherein ³²P, ¹²¹Sn or ⁹⁰Y are present in substantial amounts. Alternatively, the soluble compound of a radioisotope can be rendered insoluble, e.g., by encapsulation or immobilization in an insoluble coating or matrix. Various other metal oxides and metal phosphides are also suitable for use in the present invention.

The insoluble radioisotopes or insoluble compounds of radioisotopes may be in the form of metal or alloy particles, metal oxide particles, or polymeric particles. The size of the particles present in the coating solution may vary, as would be apparent to one skilled in the art. A representative particle size ranges from about 5 nm to about 30 μm. As a non-limiting example, ¹⁹⁸Au particles formed by wet grinding gold range from about 1 to about 10 μm in diameter are suitable for use in the present invention. In a particular embodiment of the present invention, the radioactive coating solution comprises particles of varying sizes. See, U.S. Pat. No. 4,547,407, the entire contents of which are herein incorporated by reference.

The amount of insoluble radioisotope present in the radioactive coating solution may vary, as would be understood by one skilled in the art. A representative amount has a specific activity of about 0.1 to about 5000 Ci/g.

In a particular embodiment of the present invention, the coating solution comprises 1.0 mol/L CuSO₄, 0.75 mol/L H₂SO₄, and 35 mg/L P in particulate form, suspended in solution via agitation, wherein at least a fraction of the P is ³²P. In a further embodiment of the present invention, the coating solution comprises NiSO₄ (26 g/L), NaH₂PO₂-H₂O (26 g/L), Na-acetate (34 g/L), lactic acid (18g/L), malic acid (21 g/L), and Au in particulate form, wherein at least a portion of the Au is ¹⁹⁸Au. Additional components may be added to the coating solution to vary the physical and chemical characteristics of the coating solution.

4. Composite Coating

The present invention also relates to a method of forming a radioactive coating on a substrate, which coating comprises a metal matrix and a dispersed radioactive phase. The composite coating is formed by contacting the substrate with a radioactive coating solution comprising at least one carrier metal and either an insoluble radioisotope or an insoluble compound of a radioisotope. The radioactive coating solution may have the properties described above, as one skilled in the art would appreciate. Suitable coating techniques include, but are not limited to, electroless deposition, electrodeposition, chemical vapor deposition, physical deposition, thermal spraying, or any combination thereof. Certain methods may be more suitable for certain substrates, as would be understood by one skilled in the art.

The quantity of radioactive particles deposited onto the substrate is influenced by various factors, including (1) the concentration of radioactive particles in the coating solution; (2) particle size and distribution; and (3) coating conditions. It is generally necessary to agitate the coating solution during the coating process. Substrates coated may include, but are not limited to, metals, alloys, polymers, ceramics and composites. In a particular embodiment, the substrate may be a medical device, or a component thereof, formed of metal, alloys, polymers, ceramics or composites, or combination thereof. Representative medical devices, without limitations, include catheters, guidewires, stents and brachytherapy devices. In one embodiment, the substrate is an expandable component of a catheter. In a particular embodiment, the expandable component is formed from an alloy, such as Elgiloy™.

4a. Composite Coating Formed by Electro-Deposition

In one embodiment of the method of the present invention, the radioactive composite coating is formed on the substrate by electrodeposition. The use of electrodeposition to form composite coatings is discussed in U.S. Pat. No. 5,266,181, the entire contents of which are herein incorporated by reference. More particularly, the substrate to be coated is contacted with the coating solution of the present invention for a time, at a concentration, a temperature, a cathode current density, and inter-electrode distance sufficient to electrically deposit a radioactive composite coating thereon. In some cases, it may be necessary to clean the substrate and to remove surface oxides therefrom prior to deposition of the radioactive coating. In a particular embodiment of the method of the present invention, the radioactive coating solution comprises 1.0 mol/L CuSO₄, 0.75 mol/L H₂SO₄, and a steady state concentration of 35 mg/L P in particulate form, suspended in solution via agitation, wherein at least a fraction of P is ³²P Electrodeposition conditions may vary from one coating system to another, as would be recognized by one skilled in the art. In a particular embodiment, electrodeposition of the Cu—P coating solution above is performed at a cathode current density of 18 mA/cm², an inter-electrode distance of 5 cm, at a temperature at or near 40° C. See J. W. Graydon and D. W. Kirk, “Suspension Co-deposition in Electro-twinning Cells: The Role of Hydrodynamics,” the Canadian Journal of Chemical Engineering, vol, 69 (1991) 564-570. Agitation of the insoluble radioisotope particles is necessary via stirring or alternatively via recycle flows (500-1000 mL/min) to achieve uniform deposition rates. Coating rates vary with current density, temperature and other bath parameters. Suitable coating thickness range from about 0.1 to about 20 μm, with about 5 μm generally suitable.

4b. Composite Coating Formed by Electroless Deposition

In a further embodiment of the present invention, the radioactive composite coating is formed by electroless deposition. Electroless deposition of composite coatings is reviewed in U.S. Pat. Nos. 5,232,744 and 5,389,229. More particularly, the substrate to be coated is contacted with the coating solution comprising at least one carrier metal ion, an insoluble radioisotope or insoluble compound of a radioisotope, and a reducing agent, for a time, at a concentration, at a temperature and pH sufficient to chemically deposit a radioactive composite coating thereon. Electroless deposition conditions may vary, as would be apparent to one skilled in the art. A representative electroless deposition involves contacting the substrate with a coating solution comprising from about 0.5 to about 0.5 mol/l of a metal, from about 0.1 to about 0.5 mol/l of a reducing agent and about 0.1 to about 500 g/l of particulate matter, at least a fraction of which comprises a radioactive isotope, wherein the coating solution has a pH ranging from about 4 to about 8, at a temperature of about 50 to about 95° C., and more particularly 70-90° C., for a time dependent on the particular coating thickness desired. In this embodiment, the radioisotope present in the coating solution acts to reduce the metal present therein to deposit a metal layer on the substrate surface. Thickness of the coating may vary, and range from about 0.1 to about 20 μm, and typically from about 1 to about 2 μm. Optionally, the substrate to be includes a catalytic coating layer or activating layer is coated onto the substrate prior to coating with the radioactive coating. The catalytic coating layer may be an electrolessly deposited or electrodeposited Ni coating layer, for example.

In one embodiment of the method of the present invention, the radioactive coating solution comprises NiSO₄ (26 g/L), NaH₂PO₂.H₂O (26 g/L), Na-acetate (34 g/L), lactic acid (18 g/L), malic acid (21 g/L), and Au in particulate form, wherein at least a portion of Au is present as ¹⁹⁸Au. In a further embodiment, the radioactive coating solution comprises NiSO₄ (26 g/L), NaH₂PO₂.H₂O (26 g/L), Na-acetate (34 g/L), lactic acid (18 g/L), malic acid (21 g/L), and Y₂O₃ in particulate form, wherein at least a fraction of the Y in Y₂O₃ is ⁹⁰Y.

In a still further another embodiment, the coating solution comprises NiSO₄ (26 g/L), NaH₂PO₂.H₂O (26 g/L), Na-acetate (34 g/L), lactic acid (18 g/L), malic acid (21 g/L), and a polymer phosphate in particulate form, wherein at least a fraction of the P in is ³²P. Polymers containing phosphorus are reviewed in Nakano et al. (JP#11061032). For example, Nakano describes preparation of a 2-hydroxyethyl methacrylate/tert-Bu methacrylate/ethyl methacrylate phosphorylated with phosphorus oxychloride or polyphosphoric acid to form a polymer phosphate. In one embodiment of the present invention, a portion of the P in the phosphorus oxychloride or polyphosphonic acid is ³²P, and the resulting radioactive polymer phosphate is powder processed to form a mean particle size ranging, for example, from about 5 to about 30 μM. The radioactive polymer particles are then incorporated into the coating solution. All nonsoluble particles are kept in solution by means of intensive mechanical mixing (e.g., 300 rpm).

One advantage of the composite coating solution of the present invention is the ability to separate the radioactive source from the coating solution, e.g., by filtration. Separation makes it unnecessary to treat and dispose of the entire volume of the coating solution as radioactive waste, limiting the expense of waste treatment. According to this embodiment of the coating solution of the present invention, recharging, of isotopes is permissible, providing an economic advantage.

5. Sol-Gel

The present invention also relates to radioactive sols and sol-gels, and to radioactive coatings formed via sol-gel techniques. The radioactive sol-gel of the present invention comprises an oxide and a radioisotope. Sol-gel techniques are reviewed generally in Pierre, A., Introduction to Sol-Gel Processing (1998), which is incorporated herein by reference. The radioactive sol-gel of the present invention may be formed via either colloidal or polymeric routes, resulting in either a polymeric or a colloidal radioactive sol-gel. A discussion of polymeric and colloidal gels and synthesis routes is found in C. D. E. Lakeman and D. A. Payne, Invited Review: Sol-gel Processing of Electrical and Magnetic Ceramics, Materials Chemistry and Physics, 38 (1994) 305-324, which is incorporated herein by reference.

Formation of both the colloidal and polymeric radioactive sol-gels of the present invention involves the dissolution of a metal ion, either as alkoxides or as another organometallic compound in a suitable solvent to form a fluid sol. The metal alkoxide or other organometallic compound hydrolyzes, either partially or completely, and then polymerizes, resulting in gelation and the formation of a radioactive semi-rigid gel, known as a sol gel. The radioisotope present in the sol may be either soluble or particulate (insoluble). The specific activity of this radioisotope ranges, for example, from about 0.1 to about 5000 Ci/g. Metal alkoxides suitable for use in the present invention include, but are not limited to, alkoxides of metals including silicon, boron, zirconium, titanium and aluminum. In particular, the metal alkoxide is silicon alkoxide.

In one embodiment of the present invention, a polymeric radioactive sol-gel is formed from a sol comprising a metal alkoxide and a radioisotope, which metal alkoxide hydrolyzes and then polymerizes to form a radioactive sol-gel. In a particular embodiment of the present invention, the radioactive sol-gel is formed by reacting orthophosphoric acid with silicon alkoxide, wherein at least a fraction of the P is ³²P, to form a soluble, substantially linear polymer having P—O—Si linkages. This polymer is converted to a cross-linked polymer in the presence of sufficient water.

The radioactive sol-gel may also be formed via a colloidal route. Thus, in a particular embodiment, a Fe—P—O sol-gel may be formed according to the method described by Yamaguchi et al, in IEEE Transactions on Magnetics, 25 (1989) 3321-3323, incorporated herein by reference, wherein at least a portion of the P is ³²P in the present invention.

In a particular embodiment, the radioisotope present in the sol-gel comprises an insoluble radioisotope or compound of a radioisotope. The formation of sol-gels comprising insoluble components is reviewed in Nazeri et al., Ceramic Composites by the Sol-Gel Method: A Review, Chemical Engin. Sci. Proc. 14[11-12] (1993), pp. 1-19, the contents of which are incorporated by reference. In a particular embodiment of the method, the sol comprises a metal alkoxide and an insoluble radioisotope, which metal alkoxide hydrolyzes, either partially or completely, and then polymerizes to from a radioactive sol-gel having insoluble radioisotope dispersed therein. In a further embodiment of the present invention, the sol comprises a metal alkoxide, which hydrolyzes and polymerizes to a state short of gelation, providing a partially polymerized sol which is then impregnated with the insoluble radioisotope. The impregnated sol then further polymerizes to produce a radioactive sol-gel having an insoluble radioisotope dispersed therein. In another embodiment of the present invention, a sol comprising a metal alkoxide is hydrolyzed and polymerized to form a sol-gel, which is then impregnated with the insoluble radioisotope to produce a radioactive sol-gel having insoluble radioisotope dispersed therein.

In a particular embodiment of the present invention, a sol is prepared by hydrolysis of tetra orthosilicate (TEOS) with radioactive particles (e.g., Au/¹⁹⁸Au) or (p/³²P) mixed therein. The concentration of these particles may vary as would be recognized by those skilled in the art, with a representative activity from about 0.1 to about 5000 Ci/g.

The present invention also relates to methods of forming radioactive coatings onto substrates by sol-gel techniques. In a particular embodiment of the method, the substrate to be coated is contacted with a radioactive sol comprising a metal alkoxide or an organometallic compound and a radioisotope. The sol hydrolyzes and polymerizes to produce a radioactive sol gel on the substrate. This radioactive sol-gel is then dried, and optionally subjected to high temperature treatments that (a) may remove volatile species, including but not limited to hydroxyl groups or residual organic groups; and/or (b) result in processes which produce shrinkage and removal of residual porosity, including but not limited to sintering; and/or (3) result in processes that involve phase changes, including but not limited to crystallization and chemical reactions. The dried, and optionally high temperature treated, sol-gel forms a radioactive oxide coating comprising an oxide and a radioisotope. In a particular embodiment, the sol has undergone polymerization to a certain state, short of gelation, prior to being coated onto the substrate. Put another way, a partially polymerized sol is coated onto the substrate.

In a particular embodiment of the present invention, the substrate is contacted with a radioactive sol formed by reacting orthophosphoric acid with silicon alkoxide, wherein at least a fraction of the P in the orthophosphoric acid is ³²P, as described above. Following hydrolysis and polymerization, a radioactive sol-gel is present on the article. The sol-gel is dried and optionally densified and crystallized to form a phosphorus silicon oxide coating containing ³²P.

In another embodiment, a radioactive coating is formed by spin-coating a substrate with the radioactive Fe—P—O sol described above, where the sol has an appropriate viscosity (i.e., about 80 co). The radioactive coating is then dried in air at 200° C. Following drying, an optional heat treatment may be conducted to crystallize the gel into a polycrystalline ceramic coating. For example, heating for 24 hours at 600° C. crystallizes the coating.

5a. Composite Coating Formed by Sol-Gel

The present invention also relates to a method of forming radioactive composite coating by sol-gel processes. In a particular embodiment of the method, a substrate is contacted with a radioactive sol comprising a metal alkoxide or another organometallic compound and an insoluble radioisotope. In a particular embodiment, the radioactive sol comprises hydrolyzed tetraethyl orthosilicate (TEOS) with ³²P in particulate form dispersed therein, as described above. After the substrate is coated (i.e., by dipping or spin coating) with the sol containing a radioactive dispersed phase, it is dried to form a radioactive composite coating comprising an oxide matrix and a radioactive dispersed phase. The sol used to coat may or may not have undergone polymerization to a state short of gelation. Optionally, the radioactive coating is densified and crystallized into a crystalline ceramic article. During crystallization, the dispersed phase may optionally react/combine with the silica matrix, and consequently, the radioactive material may not appear to exist as a separate dispersed phase in the crystallized ceramic coating.

In a further embodiment of the method, a sol is formed comprising a metal alkoxide or another organometallic compound, and undergoes polymerization to a state short of gelation. An insoluble radioisotope is then introduced either into the partially polymerized sol, forming a radioactive partially polymerized sol which is then coated onto a substrate. In another embodiment of the present invention, a sol is formed comprising a metal alkoxide or another organometallic compound, and coated onto a substrate to form a sol-gel. This sol-gel is then impregnated with an insoluble radioisotope. Surface coating or full impregnation of the sol-gel can be achieved using this technique. The radioactive sol-gel is then dried and optionally crystallized into a crystalline ceramic structure.

The present invention is also directed to a method of forming multiple layers of a radioactive coating or coatings onto a substrate. Coating techniques suitable for forming such layers include, without limitation, electroless deposition, electrodeposition and sol-gel methods. According to one embodiment of the method, the substrate is contacted with a first radioactive coating solution under conditions sufficient to deposit a radioactive coating thereon. Optionally, the substrate is coated with a catalytic coating layer prior to deposition of the radioactive coating layer (i.e., a Ni activation coating layer). The substrate comprising a first radioactive coating is then contacted with one or more additional radioactive coatings solutions under conditions sufficient to deposit one or more additional radioactive coating layers thereon, thereby forming a substrate two or more radioactive coating layers. This process can be repeated to provide a substrate having multiple layers of radioactive coatings. Optionally, the coated substrate is rinsed prior to being contacted with the one or more additional radioactive coating solution, and/or between deposition of these additional radioactive coatings. Optionally, one or more catalytic/activation coating layers or activating layers may be coated onto the substrate and/or between one or more of the additional radioactive coating layers.

According to another embodiment of the present invention, the substrate is coated with a radioactive sol under conditions sufficient to deposit a radioactive sol-gel coating thereon. In a particular embodiment, the radioactive sol may be at least partially polymerized. The coated substrate is then coated with one or more additional radioactive sols under conditions sufficient to deposit a one or more additional radioactive sol-gel coatings thereon. This process can be repeated to provide a substrate having multiple layers of radioactive coatings.

The multiple radioactive coating layers of the present invention may be the same or different, and may be differential, as previously explained. For example, radioactive coating layers comprising soluble radioisotopes may be present or alternate with composite radioactive coating layers having a radioactive dispersed phase, while radioactive coating layers formed by electrodeposition may be present or alternate with radioactive coating layers formed by electroless deposition or sol-gel processes, and variations thereof. The radioisotope and/or the carrier metal present in alternating radioactive coating layers may be the same or different. In one embodiment of the method, the first radioactive coating layer is different than one or more additional radioactive coatings layers. For example, the radioisotope of the first radioactive coating layer may be different than the radioisotope of one or more of the additional radioactive coatings layers. In a particular embodiment of the method, the radioisotope of the first coating layer is ¹⁹⁸Au, while the radioisotope of one or more additional coating layers is ³²P.

In one embodiment of the method of the present invention, an additional protective coating is formed over the radioactive coating or over the top radioactive coating where multiple radioactive coatings present in layers. This protective coating seals the radioactive coating and prevents dissolution of radioisotope in solution due to, for example, corrosion or abrasion. In a particular embodiment, the protective layer may formed by coating a Ni coating solution onto a radioactive coating by, for example, electrodeposition or electroless deposition. The protective layer, unlike the radioactive composite coating, does not contain radioisotope.

The invention disclosed herein also relates to radioactive coated substrates. Radioactive substrates have a variety of industrial and medical applications. It is known, for example, that radiation can be used to inhibit cell proliferation. Thus, radioactive substrates may be useful in treating a variety of diseases associated with aberrant cell proliferation, including cancer and arterial restenosis. One purpose of the present invention, therefore, is to provide radioactive substrates useful in the treatment of human and animal disease. More specifically, a particular purpose of the present invention is to provide radioactive substrates useful in the treatment of cancer and vascular disease.

In one embodiment, the present invention relates to a coated substrate comprising at least a first layer of a radioactive coating disposed on a substrate material, wherein the radioactive coating comprises at least one carrier metal and a radioisotope. The carrier metal and radioisotope can be those carrier metals and radioisotopes identified herein for use in the radioactive coating solutions of the present invention, as would be understood by one skilled in the art. In a particular embodiment, the coating comprises Ni and phosphorus, wherein at least a fraction of the phosphorus is ³²P. The coating may have a P content ranging, for example from low (1-4 weight %P) to medium (5-8 weight %P) to high (9-16 weight %P). In a particular embodiment, the fraction of P that is ³²P is about 0.01% or less. Optionally, a catalytic coating layer or activation layer is also present, interposed between the substrate and the first layer of radioactive coating. For example, a non-radioactive Ni coating may be interposed between the substrate and the first radioactive coating layer.

In a further embodiment, the present invention relates to a coated substrate comprising at least a first layer of a radioactive composite coating comprising a metal matrix and a radioactive phase dispersed therein, disposed over a substrate material. The metal matrix may be formed of those metal identified herein for use in the radioactive coating solutions of the present invention, as would be understood by one skilled in the art. Similarly, the radioactive phase may be formed of those insoluble radioisotopes or insoluble compounds of radioisotopes identified herein for use in the radioactive coating solution of the present invention, as would be understood by one skilled in the art. Optionally, a catalytic coating layer (e.g., a non-radioactive Ni coating) is also present, interposed between the substrate and the first radioactive coating layer.

The present invention is also directed to substrates comprising multiple radioactive coating layers, which coating layers may be the same or different in composition or method of deposition, or both. Optionally, one or more catalytic coating layers may be interposed between one or more of the multiple radioactive coating layers. A activation or catalytic layer may also be formed onto the substrate prior to deposition of a radioactive coating layer thereon. In one embodiment of the present invention, the first layer of radioactive coating is different from at least one or more additional layers. For example, the radioisotope of the first layer is different from the radioisotope of at least one or more additional layers. In a particular embodiment, the radioisotope of one layer of radioactive coating is ¹⁹⁸Au, while the radioisotope of one or more additional layers is ³²P. Multiple radioactive coatings layers may be deposited by electrodeposition or electroless deposition, sol-gel methods, or a combination thereof. Suitable substrates include, but are not limited to, metals, alloys, polymers, plastics, ceramics and composites.

FIG. 8B depicts a substrate comprising multiple radioactive coating layers. A nickel substrate (1) is shown having an electrodeposited Au/¹⁹⁸Au layer (2) formed thereon. An electro deposited Ni activation layer (3) if further formed on top of the Au/¹⁹⁸Au layer (2). Electrolessly deposited onto the Ni activation layer (3) is a Ni—P/³²P coating layer (4). Finally, a protective coating (5) comprising Ni—P is formed by electroless deposition onto the coated substrate.

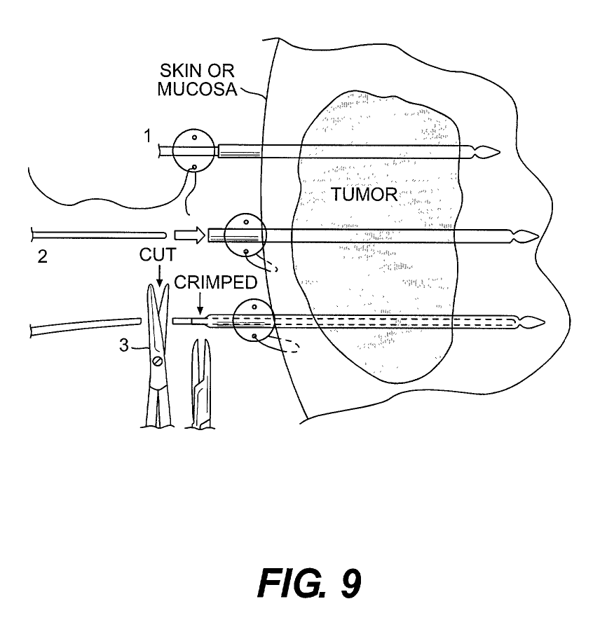

FIG. 9 depicts the conventional use of a brachytherapy to treat a tumor with local radiation. Needles coated with a radioactive isotope (10) are inserted through the skin (20) and into a tumor (30). The needles, which are fastened to the skin by a clip (40), are temporarily kept in place. Once the treatment is completed, the needles are removed.

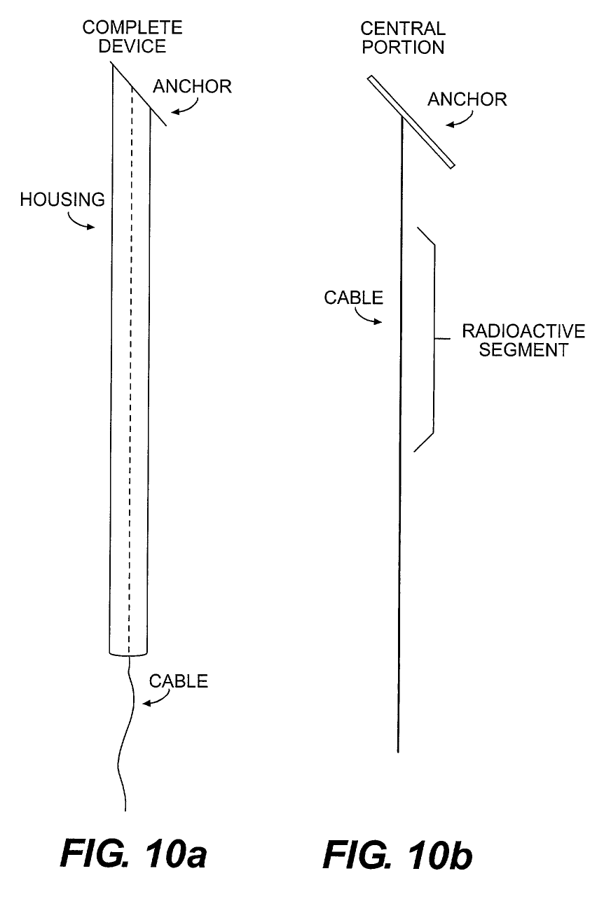

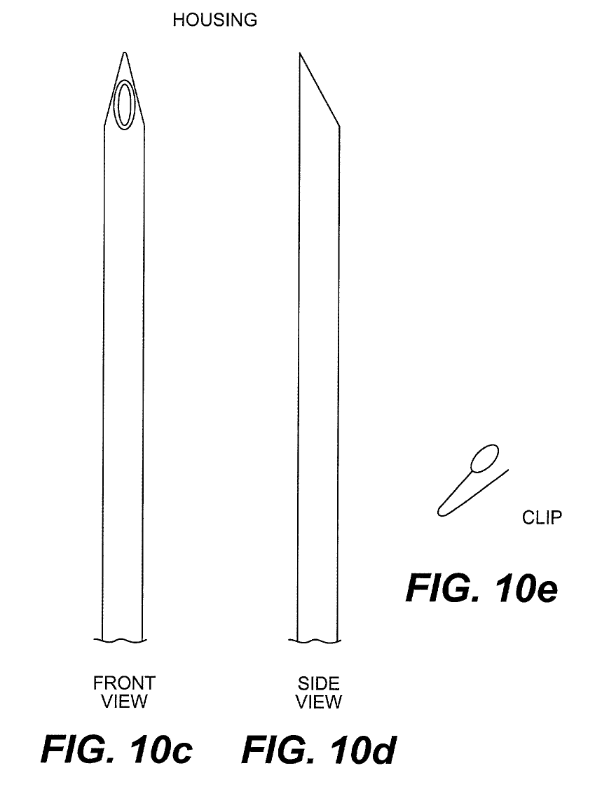

FIG. 10 is an example of a brachytherapy device according to the present invention. The device includes a central portion (50), which comprises a wire or cable (51) and an anchor (52) securely attached to the wire or cable. The wire or cable contains a radioactive segment (53), which includes at least one radioactive layer. The central portion (50) is located within a housing (60), which may contain an opening on one end (61). To treat a tumor, the complete brachytherapy device is advanced through the skin and into a tumor. The housing (60) is then withdrawn, leaving the central portion in place (50). Tension is placed on the cable (51) to deploy the anchor (52). The tension is maintained with a clip (70) that attaches on the cable (51) and fastened to the skin. The remaining cable after the clip is cut. Unlike conventional brachytherapy techniques, the radioactive segment on the cable (53) remains in the tumor for an extended period of time.

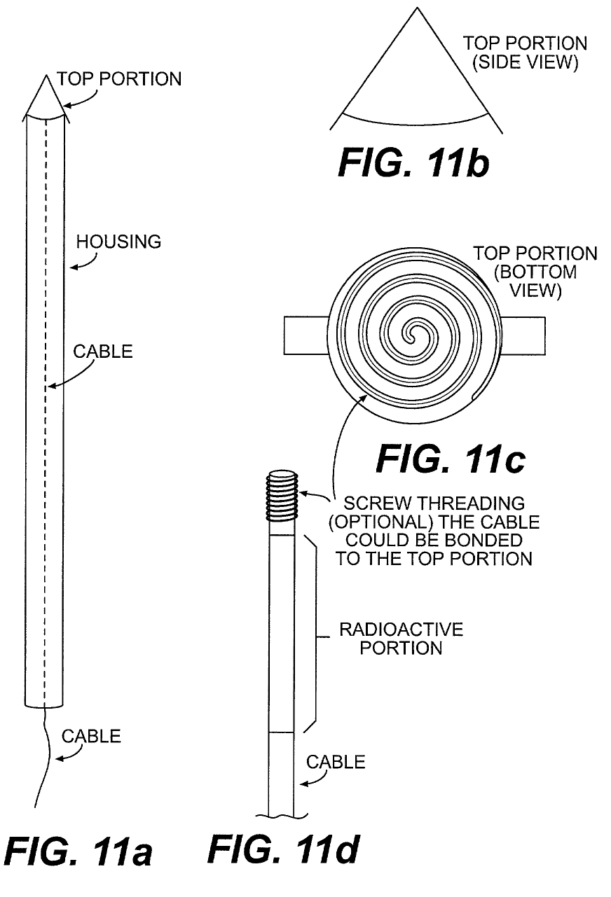

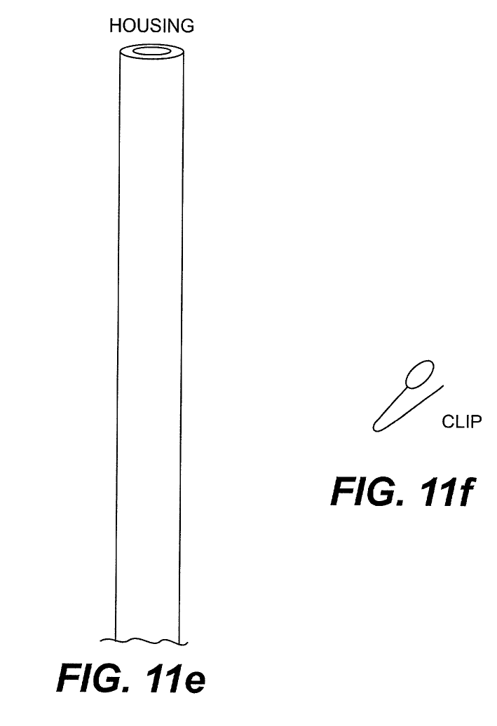

FIG. 11 is another embodiment of a brachytherapy device according to the present invention. The device includes a central portion (100), which comprises a wire or cable (110), which can be threaded at one end (120). The wire or cable contains a radioactive segment (130), which includes at least one radioactive layer. Unlike the embodiment described in FIG. 10, the top portion of this embodiment is conical with two side flaps that are used to anchor the central portion (100) into tissue. In addition, the central portion could be bonded to a top portion (200) by screw threading (210). The central portion (100) is located within a housing (300). The device is deployed in the same manner described above.

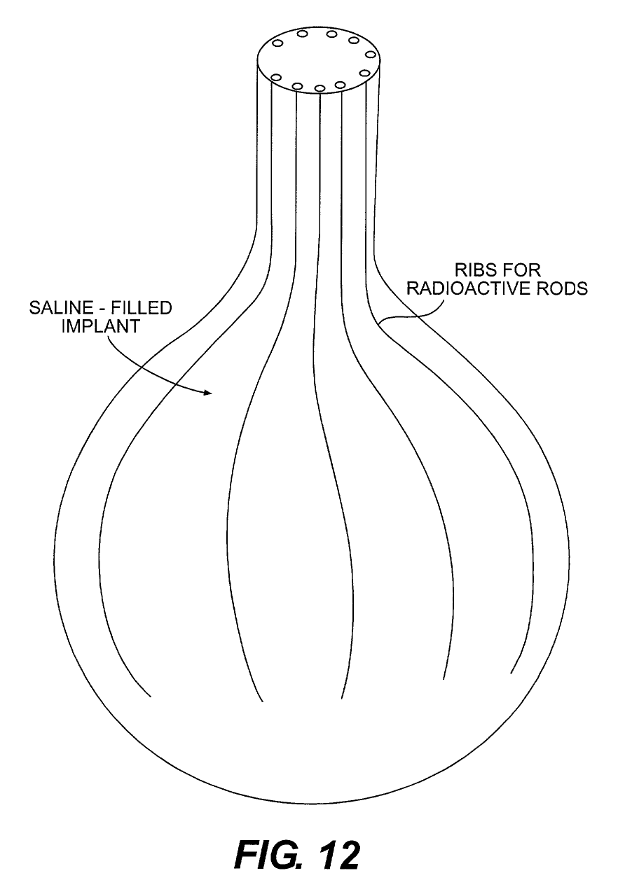

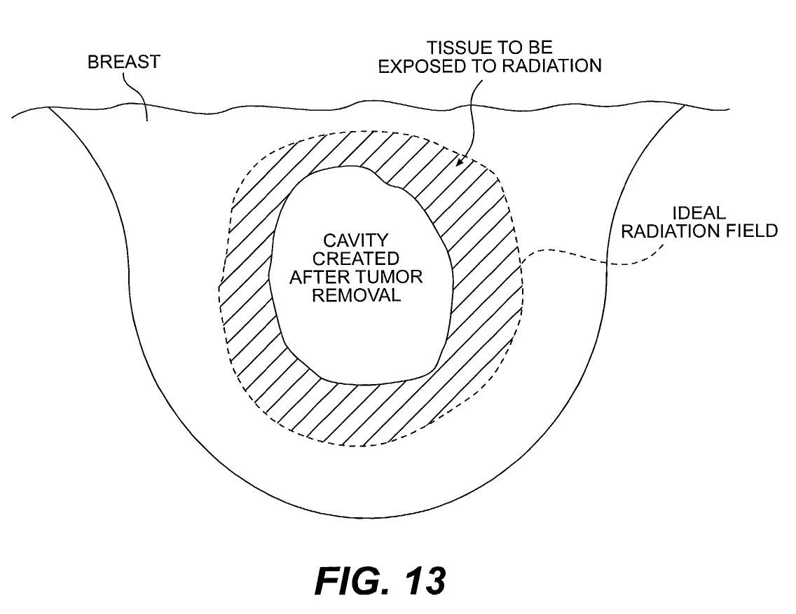

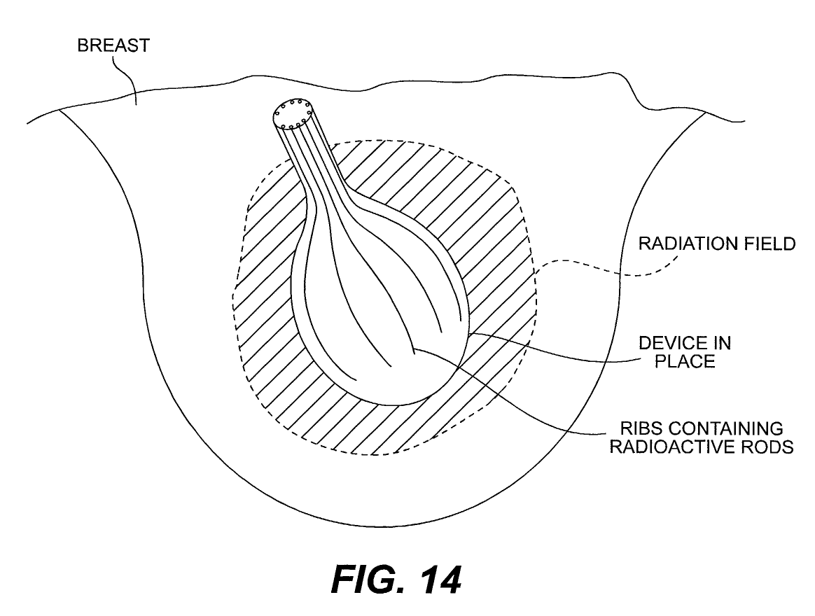

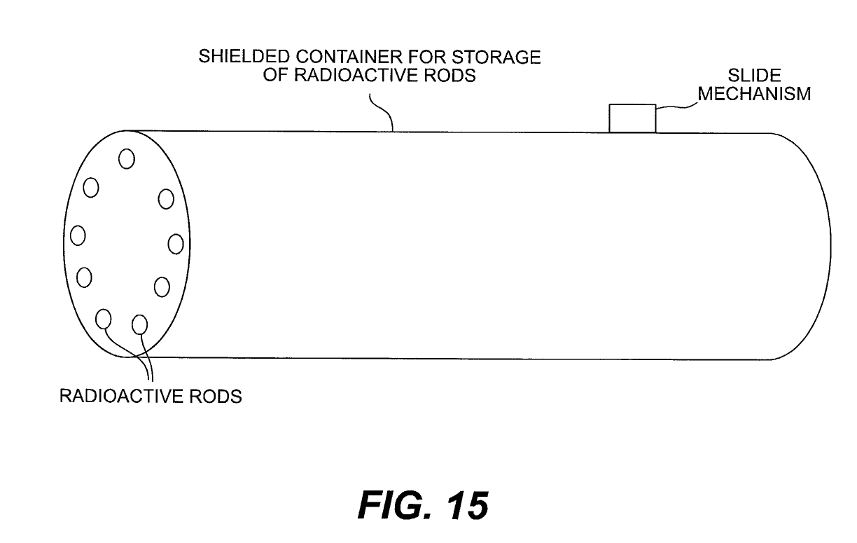

FIG. 12 is an brachytherapy device including an expandable implant containing ribs for radioactive rods. The expandable implant device is expanded by filling it with a fluid, such as saline or polymer. The device represented in FIG. 12 can be implanted into a cavity created by the removal of a tumor, in the breast, for example. FIG. 14 shows such a device in place, as well as the radiation field created by the radioactive rods placed into to the ribs of the device. Preferably, the radiation field extends within the tissue surrounding the excision cavity by not more than 3 cm, more preferably 2 cm.

The present invention also relates to the method of ion implantation as a surface modification technique to render the medical devices according to this invention radioactive. In a particular embodiment of this method, a source of ³²P is generated and accelerated to a voltage of 100 keV. (A range of voltage may be used, between 25 keV and 500 keV, typically between 50 and 150 keV). A substrate for subsequent use in a brachytherapy device, for example, is placed in the end station of an ion implanter, on a rotatable platform. This platform allows for the rotation of the device to allow for ion implantation to occur on all sides of the catheter, in order to have a controlled distribution of ³²P over the surface of the device. Grounding of the device is achieved through use of a wire, which also serves to measure the total beam current delivered to the device, to allow a direct measure of beam current. In order to activate a device to a total activity of 20 mCi, approximately 2.2×10⁻⁶ m-mole of ³²P is delivered to the device. This method advantageously provides ³²P that is embedded within approximately the top 1 micron of alloy. That is, the radioisotope is not present as a surface layer, but rather as a surface alloy that is an integral part of the substrate and therefore not subject to delamination.

In a particular embodiment, the present invention relates to a method of producing a radiation field by the above described deposition techniques, wherein the radiation field corresponds to the morphology of a tumor. According to this embodiment of the method, one or more radioactively coated brachytherapy devices can be designed or chosen based on their characteristic radiation fields to produce a radiation field correspond to the morphology of a particular tumor. The radioactively coated brachytherapy device may be the device described herein, as would be apparent to one skilled in the art.

Information on the dosimetry of the radiation fields associated with these devices can be obtained either by calculations or empirically with the measurement of the radiation field produced after the device is placed in a substance that can measure the amount of radiation at known distances from the device. The information can be used to combine different isotopes and devices, and to determine the appropriate placement of devices, and to determine the appropriate placement of the devices so that a radiation field that closely matches the morphology of a tumor can be constructed.

While the foregoing specification teaches the principles of the present invention, with examples provided for the purpose of illustration, it will be understood that the practice of the invention encompasses all of the usual variations, adaptations, and modifications, as come within the scope of the following claims and their equivalents.

What is claimed is:

1. A brachytherapy device comprising:
a substrate comprising at least one radioactive coating layer formed thereon, said radioactive coating layer having a total radioactivity that varies in at least one dimension of the device.

2. The brachytherapy device of claim 1, wherein the substrate is a hollow needle or a solid needle.

3. The brachytherapy device of claim 1, wherein the substrate is a wire, cable or rod.

4. The brachytherapy device of claim 1, wherein the at least one radioactive coating layer comprises a variable concentration of a radioisotope along said dimension.

5. The brachytherapy device of claim 1, further comprising one or more additional radioactive coating layers covering at least a portion thereof.

6. The brachytherapy device of claim 5 wherein the least one radioactive coating layer is different from at least one of the one or more additional radioactive coating layers.

7. The brachytherapy device of claim 6, wherein the least one radioactive coating layer comprises ¹⁹²Ir and said one or more additional radioactive coating layers comprises a radioisotope selected from the group consisting of ³²P, ¹⁰³Pd and ¹⁹⁸Au.

8. The brachytherapy device of claim 7, wherein one or both of the least one radioactive coating layer and the additional radioactive coating layers comprise a variable concentration of radioisotope along said dimension.

9. The brachytherapy device of claim 1, wherein the radioactive coating comprises a metal matrix and a radioactive dispersed phase.

10. The brachytherapy device of claim 1, further comprising a catalytic coating layer interposed between the substrate and the first radioactive coating layer.

11. The brachytherapy device of claim 1, further comprising one or more catalytic coating layers interposed between one or more additional radioactive coating layers.

12. A brachytherapy device comprising:
(a) a substrate comprising at least one radioactive coating layer formed thereon, said radioactive coating layer having a total radioactivity that varies in at least one dimension of the device;
(b) an anchor attached to said substrate at one end; and
(c) a clip attached to said substrate at the end opposite said anchor.

13. The brachytherapy device of claim 12, wherein said substrate is a wire, cable or rod.

14. The brachytherapy device of claim 12, wherein said anchor has a flat shape.

15. The brachytherapy device of claim 12, wherein said anchor has a conical shape with two side flaps that can attach to human tissue or a tumor.

16. The brachytherapy device of claim 15, wherein said substrate and said anchor have threaded portions which may be used to attach said anchor to said substrate.

17. A method for treating a tumor with radiation, said method comprising:
(a) advancing a brachytherapy device through the skin or mucosa and into a tumor, wherein the brachytherapy device comprises:
a central portion, and
an anchor securely attached to the central portion, said central portion containing a radioactive segment which includes at least one radioactive layer, wherein said central portion is located within a housing, which may contain an opening at one end;
(b) withdrawing the housing while leaving the central portion in the body;
(c) pulling the central portion to put tension on the central portion and to deploy the anchor such that the anchor is securely fixed within the body; and
(d) maintaining tension on the central portion by attaching a clip on the central portion and fastening said clip to the skin or mucosa.

18. The method according to claim 17, wherein said central portion is a wire, cable or rod.

19. The method according to claim 17, wherein said tumor is located in the breast or prostate.

20. A brachytherapy device comprising:
rods having at least one radioactive coating layer formed thereon; and
an inflatable device comprising openings in which the rods are placed.

21. The brachytherapy device of claim 20, wherein the radioactive coating layer has a total radioactivity that varies in at least one dimension of the device.

22. A method for treating a tumor with radiation, said method comprising:
(a) surgically removing a tumor from a human body thereby forming an excision cavity in the human body;
(b) placing a brachytherapy device in said cavity, wherein the brachytherapy device comprises rods having at least one radioactive coating layer formed thereon; and an inflatable device comprising openings in which said rods are placed.

23. The method according to claim 22, wherein said brachytherapy device produces a radioactive field that extends not more than 2 cm within the tissue surrounding the excision cavity.

24. The method according to claim 22, wherein said tumor is located in the breast, brain, head, neck, uterine or prostate.

25. A device that is implanted into a living body, said device comprising an outer layer composed of a drug delivery polymer.

26. The device of claim 25, wherein the polymer is a hydrogel.

27. The device of claim 26, wherein the polymer is loaded with a drug prior to being placed in the body.

28. The device of claim 25, wherein the outer layer comprises a porous material and a removable neck comprising a lumen, wherein said lumen is connected to the porous material, the device further comprising a drug that is infused into the lumen in the neck of the device and into the porous layer while the device is in the body.
